(19) United States
(12) Patent Application Publication
(10) Pub. No.: US 2011/0118275 A1
(43) Pub. Date: May 19, 2011

(54) OXAZOLO[4,5-c]PYRIDINE SUBSTITUTED PYRAZINE

(75) Inventors: Kristofer Ahlin, Sodertalje (SE); Per I. ARVIDSSON, Sodertalje (SE); Ulrika YNGVE, Sodertalje (SE); Fernando HUERTA, Sodertalje (SE)

(73) Assignee: ASTRAZENECA AB, Sodertalje (SE)

(21) Appl. No.: 12/945,285

(22) Filed: Nov. 12, 2010

Related U.S. Application Data
(60) Provisional application No. 61/260,984, filed on Nov. 13, 2009.

(51) Int. Cl. A61K 31/497; C07D 498/04; A61P 25/00; A61P 25/28; A61P 25/16; A61P 25/18; A61P 3/10; A61P 29/00; A61P 35/04; A61P 27/00; A61P 35/02; A61P 19/08; A61P 19/10

(52) U.S. Cl. 514/252.11; 544/357; 544/405

(57) ABSTRACT

The present invention relates to a compound of formula (I)

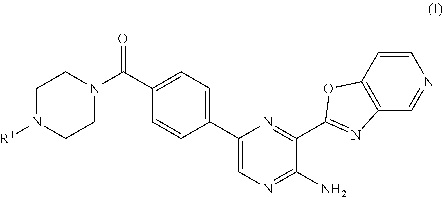

wherein
R¹ is hydrogen or methyl, or a pharmaceutically acceptable salt thereof, pharmaceutical formulations containing said compound, to the use of said active compound in therapy, as well as intermediates.

### FIELD OF THE INVENTION

The present invention relates to a compound of formula (I) or a pharmaceutically acceptable salt thereof, to pharmaceutical formulations containing said compound and to the use of said compound in therapy. Further, the present invention relates to a process for the preparation of a compound of formula (I) and to intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it (Kannoji et al, Expert Opin. Ther. Targets 2008, 12, 1443-1455).

### Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3β), or Tau phosphorylating kinase, selectively phosphorylates the microtubule associated protein Tau in neurons at sites that are hyperphosphorylated in AD brains. Hyperphosphorylated tau has lower affinity for microtubules and accumulates as paired helical filaments, which are the main components that constitute neurofibrillary tangles and neuropil threads in AD brains. This results in depolymerization of microtubules, which leads to death of axons and neuritic dystrophy. (Hooper et al., J. Neurochem. 2008, 104(6): 1433-1439). Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalitic parkinsonism, progressive supranuclear palsy, Niemann-Pick's Disease and Pick's Disease. Addition of amyloid-β to primary hippocampal cultures results in hyperphosphorylation of tau and a paired helical filaments-like state via induction of GSK3β activity, followed by disruption of axonal transport and neuronal death (Imahori and Uchida, J. Biochem. 1997, 121:179-188), while GSK3α has been postulated to regulate the production of amyloid-β itself (Phiel et al. Nature, 2003, 423, 435-439). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 1996, 93: 2719-2723). Acetyl-Co-A is critical for the synthesis of acetylcholine, a neurotransmitter with cognitive functions. Accumulation of amyloid-β is an early event in AD. GSK transgenic mice show increased levels of amyloid-β in brain. Also, PDAPP(APP^{V717F}) transgenic mice fed with Lithium show decreased amyloid-β levels in hippocampus and decreased amyloid plaque area (Su et al., Biochemistry 2004, 43: 6899-6908). Likewise, GSK3β inhibition has been shown to decrease amyloid deposition and plaque-associated astrocytic proliferation, lower tau phosphorylation, protect against neuronal cell death, and prevent memory deficincies in a double APP^{sw}-tau^{vlw} mouse model (Serenó et al, Neurobiology of Disease, 2009, 35, 359-367). Furthermore, GSK3 has been implicated in synaptic plasticity and memory function (Peineau et al., Neuron 2007, 53, 703-717; Kimura et al., PloS ONE 2008, 3, e3540), known to be impaired in AD patients. In summary, GSK3 inhibition may have beneficial effects in progression as well as the cognitive deficits associated with Alzheimer's disease and other above-referred to diseases.

### Acute Neurodegenerative Diseases

Growth factor mediated activation of the PI3K/Akt pathway has been shown to play a key role in neuronal survival. The activation of this pathway results in GSK3β inhibition. GSK3β activity is increased in cellular and animal models of neurodegeneration such as cerebral ischemia or after growth factor deprivation (Bhat et al., PNAS 2000, 97: 11074-11079). Several compounds with known GSK313 inhibitory effect has been shown to reduce infarct volume in ischemic stroke model rats. A recent publication (Koh et al, BBRC 2008, 371, 894-899) reports that GSK-3 inhibition decreased the total infarction volume and improved neurobehavioral functions by reducing ischemic cell death, inflammation, brain edema, and glucose levels, in a focal cerebral ischemia model. Thus, GSK3β inhibitors may be useful in attenuating the course of acute neurodegenerative diseases.

### Bipolar Disorders (BD)

Bipolar Disorders are characterized by manic episodes and depressive episodes. Lithium has been used to treat BD based on its mood stabilizing effects. The disadvantage of lithium is the narrow therapeutic window and the danger of overdosing that can lead to lithium intoxication. The discovery that lithium inhibits GSK3 at therapeutic concentrations has raised the possibility that this enzyme represents a key target of lithium's action in the brain (Stambolic et al., Curr. Biol. 1996, 68, 1664-1668; Klein and Melton; PNAS 1996, 93, 8455-8459; Gould et al., Neuropsychopharmacology, 2005, 30, 1223-1237). GSK3 inhibitor has been reported to reduce immobilization time in forced swim test, a model to assess on depressive behavior (O'Brien et al., J Neurosci 2004, 24, 6791-6798). GSK3 has been associated with a polymorphism found in bipolar II disorder (Szczepankiewicz et al., Neuropsychobiology. 2006, 53, 51-56) Inhibition of GSK3β may therefore be of therapeutic relevance in the treatment of BD as well as in AD patients that have affective disorders.

### Schizophrenia

Accumulating evidence implicates abnormal activity of GSK3 in mood disorders and schizophrenia. GSK3 is involved in signal transduction cascades of multiple cellular processes, particularly during neural development. (Kozlovsky et al., Am. J. Psychiatry, 2000, 157, 831-833) states that GSK3β levels were 41% lower in the schizophrenic patients than in comparison subjects. This study reports that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 1998, 9, 1379-1383). Atypical antipsychotic such as olanzapine, clozapine, quetiapine, and ziprasidone, inhibits GSK3 by increasing ser9 phosphorylation discussing that antipsychotics may exert their beneficial effects via GSK3 inhibition (Li X. et al., Int. J. of Neuropsychopharmacol, 2007, 10: 7-19).

### Diabetes

Type 2 diabetes mellitus is characterized by insulin resistance and β-cell failure. Insulin stimulates glycogen synthesis in skeletal muscles via dephosphorylation and thus activation of glycogen synthase and therefore increased glucose disposal. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2): 263-71). Inhibition of GSK3 increases the activity of glycogen synthase thereby decreasing glucose levels by its conversion to glycogen. In animal models of diabetes, GSK3 inhibitors lowered plasma glucose levels up to 50% (Cline et al., Diabetes, 2002, 51: 2903-2910; Ring et al., Diabetes 2003, 52: 588-595). Moreover, results obtained by using haploinsufficient GSK3β mice on a diabetic background recites that reduced GSK3β activity also protects from β-cell failure (Tanabe et al., PloS Biology, 2008, 6(2):307-318 GSK3 inhibition may therefore be of therapeutic relevance in the treatment of Type I and Type II diabetes to enhance insulin sensitivity and reduce β-cell failure and therefore also relevant therapy to reduce diabetic complications like diabetic neuropathy.

### Alopecia

GSK3 phosphorylates and degrades β-catenin. β-Catenin is an effector of the pathway for keratonin synthesis. β-Catenin stabilization may be lead to increase hair development. Mice expressing a stabilized β-catenin by mutation of sites phosphorylated by GSK3 undergo a process resembling de novo hair morphogenesis (Gat et al., Cell, 1998, 95, 605-14)). The new follicles formed sebaceous glands and dermal papilla, normally established only in embryogenesis. Thus, GSK3 inhibition may offer treatment for a variety of indications that lead to alopecia.

### Inflammatory Disease

The discovery that GSK3 inhibitors provide anti-inflammatory effects has raised the possibility of using GSK3 inhibitors for therapeutic intervention in inflammatory diseases. (Martin et al., Nat. Immunol. 2005, 6, 777-784; Jope et al., Neurochem. Res. 2007, 32, 577-595). Inflammation is a common feature of a broad range of conditions including Alzheimer's Disease and mood disorders. A recent publication (Kitazawa et al, Ann. Neurol. 2008; 64; 15-24) discuss that GSK3β may play a role in inclusion body myositis (IBM).

### Cancer

GSK3 is over expressed in ovarian, breast and prostate cancer cells and recent data suggests that GSK3β may have a role in contributing to cell proliferation and survival pathways in several solid tumor types. GSK3 plays an important role in several signal transduction systems which influence cell proliferation and survival such as WNT, PI3 Kinase and NFkB. GSK3β deficient MEFs indicate a crucial role in cell survival mediated NFkB pathway (Ougolkov A V and Billadeau D D., Future Oncol. 2006 February; 2(1): 91-100.). Thus, GSK3 inhibitors may inhibit growth and survival of solid tumors, including pancreatic, colon and prostate cancer.

Growth control of multiple myeloma cells has been demonstrated through inhibition of GSK3 (Zhou et al 2008 Leuk. Lymphoma, 48, 1946-1953). A recent publication (Wang et al, Nature 2008, 455, 1205-1209) discuss that GSK3 inhibition was efficacious in a murine model of MLL leukemia. Thus, GSK3 inhibitors may also inhibit growth and survival of hematological tumors, including multiple myeloma.

### Glaucoma

There is a possibility of using GSK3 inhibitors for therapeutic treatment of glaucoma. Elevated intraocular pressure (IOP) is the most significant risk factor for the development of glaucoma, and current glaucoma therapy focuses on reducing IOP, either by reducing aqueous humor production or by facilitating aqueous humor outflow. Recently published expression profiling experiments (Wang et al., J. Clin. Invest. 2008, 118, 1056-1064) have revealed that the soluble WNT antagonist sFRP-1 is over expressed in ocular cells from glaucoma patients relative to control subjects. A functional link between WNT signaling pathways and glaucoma was provided through experiments in which addition of recombinant sFRP-1 to ex vivo-cultured human eye anterior segments resulted in a decrease in aqueous humor outflow; in addition, in vivo experiments in mice demonstrated that over expression of sFRP-1 in ocular tissues resulted in increases in intraocular pressure, an effect that was antagonized by a small-molecule GSK3 inhibitor. Taken together, the results reported by Wang et al. (2008) states that activation of WNT signaling via inhibition of GSK3 may represent a novel therapeutic approach for lowering intraocular pressure in glaucoma.

### Pain

A recent publication (WO2008/057933) reports that GSK3beta inhibitors may play a role in the treatment of pain, particularly neuropathic pain, by modulation of glycogenolysis or glycolysis pathways.

### Bone-Related Disorders and Conditions

Genetic studies have established a link between bone mass in humans and Wnt signaling (Gong et al., Am. J. Hum. Genet. 1996, 59, 146-51, Little et al., N. Engl. J. Med., 2002, 347, 943-4). Genetic and pharmacological manipulations of Wnt signaling in mice have since then confirmed the central role of this pathway in regulating bone formation. Of the pathways activated by Wnts, it is signaling through the canonical (i.e., Wnt/β-catenin) pathway that increases bone mass through a number of mechanisms including renewal of stem cells, stimulation of pre-osteoblast replication, induction of osteoblastogenesis, and inhibition of osteoblast and osteocyte apoptosis. Therefore, enhancing Wnt pathway signaling with GSK3 inhibitors alone or in combination with a suitable device could be used for the treatment of bone-related disorders, or other conditions which involve a need for new and increased bone formation for example osteoperosis (genetic, iatrogenic or generated through aging/hormone imbalance), fracture repair as a result of injury or surgery, chronic-inflammatory diseases that result in bone loss such as for example rheumatoid arthritis, cancers that lead to bone lesions, such as for example cancers of the breast, prostate and lung, multiple myeloma, osteosarcoma, Ewing's sarcoma, chondrosarcoma, chordoma, malignant fibrous histiocytoma of the bone, fibrosarcoma of the bone, cancer induced bone disease, iatrogenic bone disease, benign bone disease and Paget's disease.

### Regenerative Medicine

Stem-cell expansion and differentiation are required for self-renewal and maintenance of tissue homeostasis and repair. The β-catenin-mediated canonical Wnt signaling pathway has been reported to be involved in controlling stem differentiation (Pinto et al., Exp. Cell Res., 2005, 306, 357-63). A physiological Wnt response may be essential for the regeration of damaged tissues. GSK3 inhibitors by enhancing Wnt signaling may be useful to modulate stem cell function to enhance tissue generation ex vivo or in vivo in diseases associated with tissue damage or reduced tissue repair.

### DETAILED DESCRIPTION OF THE INVENTION

The object of the present invention is to provide a compound having a high GSK3 inhibiting potency as well having good selectivity against different kinases including AXL.

Accordingly, the present invention provides a compound of formula (I), or a pharmaceutically acceptable salt thereof

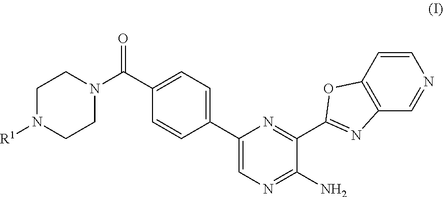

wherein R¹ is hydrogen or methyl.

One aspect of the present invention relates to a compound of formula (I), or a pharmaceutically acceptable salt thereof, wherein R¹ is methyl.

The present invention provides a compound, or a pharmaceutically acceptable salt thereof, selected from
4-(5-amino-6-(oxazolo[4,5-c]pyridin-2-yl)pyrazin-2-yl)phenyl)(4-methylpiperazin-1-yl)methanone or
(4-(5-amino-6-(oxazolo[4,5-c]pyridin-2-yl)pyrazin-2-yl)phenyl)(piperazin-1-yl)methanone.

One embodiment of the present invention is (4-(5-amino-6-(oxazolo[4,5-c]pyridin-2-yl)pyrazin-2-yl)phenyl)(4-methylpiperazin-1-yl)methanone, or a pharmaceutically acceptable salt thereof
Another embodiment of the present invention is 4-(5-amino-6-(oxazolo[4,5-c]pyridin-2-yl)pyrazin-2-yl)phenyl)(4-methylpiperazin-1-yl)methanone in the free base form.

In yet still another embodiment of the present invention is 4-(5-amino-6-(oxazolo[4,5-c]pyridin-2-yl)pyrazin-2-yl)phenyl)(4-methylpiperazin-1-yl)methanone hydrochloride; (4-(5-Amino-6-(oxazolo[4,5-c]pyridin-2-yl)pyrazin-2-yl)phenyl)(4-methylpiperazin-1-yl)methanone hydrobromide; (4-(5-Amino-6-(oxazolo[4,5-c]pyridin-2-yl)pyrazin-2-yl)phenyl)(4-methylpiperazin-1-yl)methanone mesylate; 4-(5-Amino-6-(oxazolo[4,5-c]pyridin-2-yl)pyrazin-2-yl)phenyl)(4-methylpiperazin-1-yl)methanone sulphate; 4-(5-Amino-6-(oxazolo[4,5-c]pyridin-2-yl)pyrazin-2-yl)phenyl)(4-methylpiperazin-1-yl)methanone maleate or (4-(5-Amino-6-(oxazolo[4,5-c]pyridin-2-yl)pyrazin-2-yl)phenyl)(4-methylpiperazin-1-yl)methanone tosylate.

Still other pharmaceutically acceptable salts useful in accordance with the invention and methods of preparing these salts may be found in, for example, Remington's Pharmaceutical Sciences (18th Edition, Mack Publishing Co.).

### INTERMEDIATES

The present invention also relates to intermediates for the end products, the compounds of formula (I) as defined herein.

One aspect of the invention is a compound of formula (IV)

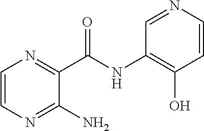

One aspect of the invention is a compound of formula (V)

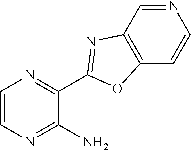

One aspect of the invention is a compound of formula (VI)

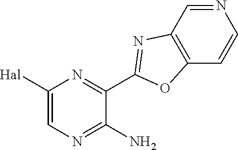

wherein Hal is chlorine, bromine or iodine. In one embodiment, Hal is bromine.

One aspect of the invention is a compound of formula (IV)

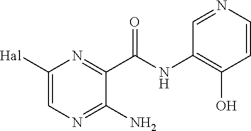

wherein Hal is chlorine, bromine or iodine. In one embodiment, Hal is bromine.

These intermediates are represented by, but not limited to, the following:
3-Amino-N-(4-hydroxypyridin-3-yl)pyrazine-2-carboxamide,
3-(Oxazolo[4,5-c]pyridin-2-yl)pyrazin-2-amine,
5-Bromo-3-(oxazolo[4,5-c]pyridin-2-yl)pyrazin-2-amine,
tert-Butyl 4-(4-(5-amino-6-(oxazolo[4,5-c]pyridin-2-yl)pyrazin-2-yl)benzoyl)piperazine-1-carboxylate
and 6-Bromo-3-amino-N-(4-hydroxypyridin-3-yl)pyrazine-2-carboxamide.

Listed below are definitions of various terms used in the specification and claims to describe the present invention.

The term “halogen” or “Hal” refers to fluorine, chlorine, bromine and iodine.

Compounds of formula (I) in which any nitrogen atom not linked to the carboxy group within said heterocyclic ring is optionally oxidised to N⁺O⁻ are also comprised in the invention.

The present invention further includes isotopically-labeled compounds of the invention. An “isotopically” or “radio-labeled” compound is a compound of the invention where one or more atoms are replaced or substituted with an atom having an atomic mass or mass number different from the atomic mass or mass number typically found in nature (i.e., naturally occurring). Suitable stable or radioactive nuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹³C, ¹⁴C, ¹²⁵I, ³⁵S and ⁸²Br. In other embodiments the radionuclide is selected from the group consisting of ¹³C and ¹⁴C.

The present invention also relates to the use of a compound of formula (I) as hereinbefore defined.

Salts for use in pharmaceutical formulations will be pharmaceutically acceptable salts, but other salts may be useful in the production of the compounds of formula (I).

### Pharmaceutical Formulations

According to one aspect of the present invention there is provided a pharmaceutical formulation comprising the compound of formula (I) or a pharmaceutically acceptable salt thereof, for use in the prevention and/or treatment of conditions associated with glycogen synthase kinase-3.

The formulation used in accordance with the present invention may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream, for rectal administration as a suppository and for local administration in a body cavity.

Suitable daily doses of the compound of formula (I) or pharmaceutically acceptable salts thereof in the treatment of a mammal, including human, are approximately 0.01 to 250 mg/kg bodyweight at per oral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

The compound of formula (I) or a pharmaceutically acceptable salt thereof, may be used on its own but will usually be administered in the form of a pharmaceutical formulation in which the active ingredient is in association with pharmaceutically acceptable diluents, excipients and/or inert carrier known to a person skilled in the art. Dependent on the mode of administration, the pharmaceutical formulation may comprise from 0.05 to 99% w (percent by weight), for example from 0.10 to 50% w, of active ingredient, all percentages by weight being based on total composition.

The invention further provides a process for the preparation of a pharmaceutical formulation of the invention which comprises mixing of the compound of formula (I) or a pharmaceutically acceptable salt thereof, as hereinbefore defined, with pharmaceutically acceptable diluents, excipients and/or inert carriers.

A suitable pharmaceutically acceptable salt of the compound of formula (I) useful in accordance to the invention is, for example, an acid-addition salt, obtained with, for example an inorganic acid such as hydrochloric acid, hydrobromic acid, sulfuric acid or orthophosphoric acid, or organic acid such as acetic acid, maleic acid, fumaric acid, methane sulfonic acid, ethane sulfonic acid, p-toluenesulfonic acid, 2-naphtalene sulfonic acid and 1,5-naphtalene sulfonic acid. (further examples see Stahl and Wermuth, Handbook of Pharmaceutical Salts: Properties, Selection and Use (Wiley-VCH, 2002).

As used herein, “a free base or a pharmaceutically acceptable salt” refer to ansolvates, to including anhydrates and desolvated solvates, and solvates, including hydrates. As used herein, “pharmaceutically acceptable salts” refer to derivatives of the disclosed compounds wherein the parent compound is modified by making salts or co-crystals thereof. It is to be understood that the present invention encompasses all such forms that possess GSK3 inhibitory activity.

In a salt proton transfer occurs between the compound of formula (I) and the counter ion of the salt. However, in some cases proton transfer may not be complete and the solid is not therefore a true salt. In such cases the compound of formula (I) and the “co-former” molecules in the solid primarily interact through non-ionic forces such as hydrogen bonding. It is accepted that the proton transfer is in fact a continuum, and can change with temperature, and therefore the point at which a salt is better described as a co-crystal can be somewhat subjective.

Where an acid or base co-former is a solid at room temperature and there is no or only partial proton transfer between the compound of formula (I) and such an acid or base co-former, a co-crystal of the co-former and compound of formula (I) may result rather than a salt. All such co-crystal forms of the compound of formula (I) are encompassed by the present invention. The definition of the co-former acid or base being a solid at room temperature is intended to distinguish co-crystals from solvates (including hydrates), of the compound of formula (I). However, a salt or co-crystal of a compound of formula (I) may itself form solvates (including hydrates). It is to be understood that all such solvates of a salt or co-crystal are also encompassed by the present invention.

The compound of formula (I) may form a mixture of its salt and co-crystal forms. It is also to be understood that the present invention encompasses salt/co-crystal mixtures of the compound of formula (I), as well as any solvates (including hydrates) thereof.

Salts and co-crystals may be characterised using well known techniques, for example X-ray powder diffraction, single crystal X-ray diffraction (for example to evaluate proton position, bond lengths or bond angles), solid state ¹HNMR, (to evaluate for example, C, N or P chemical shifts) or spectroscopic techniques (to measure for example, O—H, N—H or COOH signals and IR peak shifts resulting from hydrogen bonding).

### Medical Uses

It has been found that the compound of formula (I) defined in the present invention, is well suited for inhibiting glycogen synthase kinase-3 (GSK3). Accordingly, said compound of the present invention is expected to be useful in the prevention and/or treatment of conditions associated with glycogen synthase kinase-3 activity, i.e. the compounds may be used to produce an inhibitory effect of GSK3 in mammals, including human, in need of such prevention and/or treatment.

GSK3 is highly expressed in the central and peripheral nervous system and in other tissues. Thus, it is expected that the compound of the invention is well suited for the prevention and/or treatment of conditions associated with glycogen synthase kinase-3 in the central and peripheral nervous system. In particular, the compound of the invention is expected to be suitable for prevention and/or treatment of conditions associated with cognitive disorder(s) or indications with deficit(s) in cognition such as: dementia; incl. pre-senile dementia (early onset Alzheimer's Disease); senile dementia (dementia of the Alzheimer's type); Alzheimer's Disease (AD); Familial Alzheimer's disease; Early Alzheimer's disease; mild to moderate dementia of the Alzheimer's type; delay of disease progression of Alzheimer's Disease; neurodegeneration associated with Alzheimer's disease, Mild Cognitive Impairment (MCI); Amnestic Mild Cognitive Impairment (aMCI); Age-associated Memory Impairment (AAMI); Lewy body dementia; vascular dementia (VD); HIV-dementia; AIDS dementia complex; AIDS—Neurological Complications; Frontotemporal dementia (FTD); Frontotemporal dementia Parkinson's Type (FTDP); dementia pugilistica; dementia due to infectious agents or metabolic disturbances; dementia of degenerative origin; dementia—Multi-Infarct; memory loss; cognition in Parkinson's Disease; cognition in multiple sclerosis; cognition deficits associated with chemotherapy; Cognitive Deficit in Schizophrenia (CDS); Schizoaffective disorders including schizophrenia; Age-Related Cognitive Decline (ARCD); Cognitive Impairment No Dementia (CIND); Cognitive Deficit arising from stroke or brain ischemia; Congenital and/or development disorders; progressive supranuclear palsy (PSP); amyotrophic lateral sclerosis (ALS); corticobasal degeneration (CBD); traumatic brain injury (TBI); postencephalitic parkinsonism; Pick's Disease; Niemann-Pick's Disease; Down's syndrome; Huntington's Disease; Creurtfeld-Jacob's disease; prion diseases; multiple sclerosis (MS); motor neuron diseases (MND); Parkinson's Disease (PD); β-amyloid angiopathy; cerebral amyloid angiopathy; Trinucleotide Repeat Disorders; Spinal Muscular Atrophy; Friedreich's Ataxia; Neuromyelitis Optica; Multiple System Atrophy; Transmissible Spongiform Encephalopathies; Attention Deficit Disorder (ADD); Attention Deficit Hyperactivity Disorder (ADHD); Bipolar Disorder (BPD) including acute mania, bipolar depression, bipolar maintenance; Major Depressive Disorders (MDD) including depression, major depression, mood stabilization, dysthymia; agnosia; aphasia; apraxia; apathy.

One embodiment of the invention relates to the prevention and/or treatment of Alzheimer's Disease, especially the use in the delay of the disease progression of Alzheimer's Disease.

Other embodiments of the invention relate to the prevention and/or treatment of disorders selected from the group consisting of attention deficit disorder (ADD), attention deficit hyperactivity disorder (ADHD) and affective disorders, wherein the affective disorders are Bipolar Disorder including acute mania, bipolar depression, bipolar maintenance, major depressive disorders (MDD) including depression, major depression, mood stabilization, schizoaffective disorders including schizophrenia, and dysthymia.

Other aspects of the compound of the invention is its use for treatment of Type I diabetes, Type II diabetes, diabetic neuropathy; pain incl. neuropathic pain, nociceptive pain, chronic pain, pain associated with cancer, pain associated with rheumatic disease; alopecia; glaucoma; inflammatory diseases; incl. inclusion body myositis (IBM); pemphigus vulgaris.

Another aspect of the compound of the invention is its use for treatment of benign or malignant tumours incl. non-solid tumours such as leukaemia including MLL leukemia; myeloma including multiple myeloma; or lymphoma; and solid tumours, for example bile duct, bone, bladder, brain/CNS, breast, colorectal, endometrial, gastric, head and neck, hepatic, lung particularly, non-small-cell lung, neuronal, oesophageal, ovarian, pancreatic, prostate, renal, skin, testicular, thyroid, uterine and vulval cancers.

Yet another aspect of the compound of the invention is its use for treatment of bone related effects of specific cancers for example breast, prostate, lung cancer, multiple myeloma, osteosarcoma, Ewing's sarcoma, chondrosarcoma, chordoma, malignant fibrous histiocytoma of bone, fibrosarcoma of bone, cancer induced bone disease and iatrogenic bone disease.

A further aspect of the compound of the invention is its use for treatment of osteoporosis (genetic, iatrogenic or generated through aging/hormone imbalance), fracture repair as a result of injury or surgery, chronic-inflammatory diseases that result in bone loss such as for example rheumatoid arthritis, cancers that lead to bone lesions, such as for example cancers of the breast, prostate and lung, multiple myeloma, osteosarcoma, Ewing's sarcoma, chondrosarcoma, chordoma, malignant fibrous histiocytoma of the bone, fibrosarcoma of the bone, cancer induced bone disease, iatrogenic bone disease, benign bone disease and Paget's disease, for promoting bone formation, increasing bone mineral density, reducing the rate of fracture and/or increasing the rate of fracture healing, increasing cancellous bone formation and/or new bone formation.

The present invention relates also to the use of the compound of formula (I) as defined in the present invention in the manufacture of a medicament for the prevention and/or treatment of conditions associated with glycogen synthase kinase-3.

The invention also provides for a method of treatment and/or prevention of conditions associated with glycogen synthase kinase-3 comprising administering to a mammal, including human in need of such treatment and/or prevention a therapeutically effective amount of the compound of formula (I) as as defined in the present invention.

The dose required for the therapeutic or preventive treatment of a particular disease will necessarily be varied depending on the host treated, the route of administration and the severity of the illness being treated.

For veterinary use the amounts of different components, the dosage form and the dose of the medicament may vary and will depend on various factors such as, for example the individual requirement of the animal treated.

In the context of the present specification, the term “therapy” also includes “prevention” unless there are specific indications to the contrary. The terms “therapeutic” and “therapeutically” should be construed accordingly.

In the context of the present specification, the term “disorder” also includes “condition” unless there are specific indications to the contrary.

Another aspect of the invention is wherein a compound of formula (I) or a pharmaceutically acceptable salt thereof as defined herein, or a pharmaceutical composition or formulation comprising a combination comprising such a compound of formula (I) is administered, concurrently, simultaneously, sequentially, separately or adjunct with another pharmaceutically active compound or compounds selected from the following:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine; and equivalents and pharmaceutically active isomer(s) and metabolite(s) thereof.

(ii) atypical antipsychotics including for example quetiapine; and pharmaceutically active isomer(s) and metabolite(s) thereof.

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone; and equivalents and pharmaceutically active isomer(s) and metabolite(s) thereof.

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam; and equivalents and pharmaceutically active isomer(s) and metabolite(s) thereof.

(v) anticonvulsants including for example carbamazepine, clonazepam, ethosuximide, felbamate, fosphenyloin, gabapentin, lacosamide, lamotrogine, levetiracetam, oxcarbazepine, phenobarbital, phenyloin, pregabaline, rufinamide, topiramate, valproate, vigabatrine, zonisamide; and equivalents and pharmaceutically active isomer(s) and metabolite(s) thereof.

(vi) Alzheimer's therapies including for example donepezil, rivastigmine, galantamine, memantine; and equivalents and pharmaceutically active isomer(s) and metabolite(s) thereof.

(vii) Parkinson's therapies including for example levodopa, dopamine agonists such as apomorphine, bromocriptine, cabergoline, pramipexol, ropinirole, and rotigotine, MAO-B inhibitors such as selegeline and rasagiline, and other dopaminergics such as tolcapone and entacapone, A-2 inhibitors, dopamine reuptake inhibitors, NMDA antagonists, Nicotine agonists, and inhibitors of neuronal nitric oxide synthase; and equivalents and pharmaceutically active isomer(s) and metabolite(s) thereof.

(viii) migraine therapies including for example almotriptan, amantadine, bromocriptine, butalbital, cabergoline, dichloralphenazone, dihydroergotamine, eletriptan, frovatriptan, lisuride, naratriptan, pergolide, pizotiphen, pramipexole, rizatriptan, ropinirole, sumatriptan, zolmitriptan; and equivalents and pharmaceutically active isomer(s) and metabolite(s) thereof.

(ix) stroke therapies including for example thrombolytic therapy with eg activase and desmoteplase, abciximab, citicoline, clopidogrel, eptifibatide, minocycline; and equivalents and pharmaceutically active isomer(s) and metabolite(s) thereof.

(x) urinary incontinence therapies including for example darafenacin, falvoxate, oxybutynin, propiverine, robalzotan, solifenacin, tolterodine; and equivalents and pharmaceutically active isomer(s) and metabolite(s) thereof.

(xi) neuropathic pain therapies including lidocain, capsaicin, and anticonvulsants such as gabapentin, pregabalin, and antidepressants such as duloxetine, venlafaxine, amitriptyline, klomipramine; and equivalents and pharmaceutically active isomer(s) and metabolite(s) thereof.

(xii) nociceptive pain therapies including paracetamol, NSAIDS and coxibs, such as celecoxib, etoricoxib, lumiracoxib, valdecoxib, parecoxib, diclofenac, loxoprofen, naproxen, ketoprofen, ibuprofen, nabumeton, meloxicam, piroxicam and opioids such as morphine, oxycodone, buprenorfin, tramadol; and equivalents and pharmaceutically active isomer(s) and metabolite(s) thereof.

(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem; and equivalents and pharmaceutically active isomer(s) and metabolite(s) thereof.

(xiv) mood stabilizers including for example carbamazepine, divalproex, gabapentin, lamotrigine, lithium, olanzapine, quetiapine, valproate, valproic acid, verapamil; and equivalents and pharmaceutically active isomer(s) and metabolite(s) thereof.

Such combination products employ the compound of this invention within the dosage range described herein and the other pharmaceutically active compound or compounds within approved dosage ranges and/or the dosage described in the publication references.

In one embodiment of the invention the combination comprises the group of compounds (a) and (b) as defined below:

(a) a first therapeutic agent, which is a GSK3 inhibitor and (b) a second therapeutic agent, which is an antipsychotic selected from:

(a) (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) quetiapine;

(a) a first therapeutic agent, which is a GSK3 inhibitor and (b) a second therapeutic agent, which is a an α7-nicotinic agonist selected from: (a) (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) ((−)-spiro[1-azabicyclo[2.2.2]octane-3,2′-(2′,3′-dihydrofuro[2,3-B]pyridine)], or a pharmaceutically acceptable salt thereof;

(a) (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) (R)-5-(5-(morpholinomethyl)furan-3-yl)-3H-1′-azaspiro[furo[2,3-b]pyridine-2,3′-bicyclo[2.2.2]octane], or a pharmaceutically acceptable salt thereof;

The combination may employ any alpha-7 agonist, including but not limited to those disclosed in U.S. Pat. Nos. 6,110,914 and 6,569,865; and pending US Application 2008-0139600 (Al), WO96/06098, WO99/03859, WO00/42044, WO01/060821, WO02/096912, WO03/087103, WO2005/030777, WO2005/030778 and WO2007/133155.

(a) a first therapeutic agent, which is a GSK3 inhibitor and (b) a second therapeutic agent, which is a an α4β2-neuronal nicotinic agonist selected from:

(a) (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) (2S)-(4E)-N-methyl-5-[3-(5-isopropoxypyridin)yl]-4-penten-2-amine, or a pharmaceutically acceptable salt thereof;

(a) (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) 3-(5-chloro-2-furoyl)-3,7-diazabicyclo[3.3.0]octane, or a pharmaceutically acceptable salt thereof;

α4β2-neuronal nicotinic agonist useful in the combination of the present invention are those described in U.S. Pat. No. 6,603,011, U.S. Pat. No. 6,958,399 and WO/2008/057938, which are hereby incorporated by reference. Particular nicotinic agonists are compounds N-methyl-5-[3-(5-isopropoxypyridin)yl]-4-penten-2-amine, (4E)-N-methyl-5-[3-(5-isopropoxypyridin)yl]-4-penten-2-amine and (2S)-(4E)-N-methyl-5-[3-(5-isopropoxypyridin)yl]-4-penten-2-amine, 3-(5-chloro-2-furoyl)-3,7-diazabicyclo[3.3.0]octane, metabolites or prodrugs and pharmaceutically-acceptable salts, solvates or solvated salts of any of the foregoing. The preparation of these compounds is described in said US patents.

(a) a first therapeutic agent, which is a GSK3 inhibitor and (b) a second therapeutic agent, which is a BACE inhibitor.
(a) a first therapeutic agent, which is the GSK3 inhibitor (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) a second therapeutic agent, which is a BACE inhibitor.

Drugs useful in the combination of the present invention are those that reduce or block BACE activity should therefore reduce Aβ levels and levels of fragments of Aβ in the brain, and thus slow the formation of amyloid plaques and the progression of AD or other maladies involving deposition of Aβ or fragments thereof.

(a) a first therapeutic agent, which is a GSK3 inhibitor and (b) a second therapeutic agent, which is a H3 antagonist.
(a) a first therapeutic agent, which is the GSK3 inhibitor (4-(5-amino-6-(oxazolo[4,5-c]pyridin-2-yl)pyrazin-2-yl)phenyl)(4-methylpiperazin-1-yl)methanone, or a pharmaceutically acceptable salt thereof and (b) a second therapeutic agent, which is a H3 antagonist.

The histamine H3 receptor has been shown to regulate the release of pro-cognitive neurotransmitters, such as, for example, histamine and acetylcholine. Some histamine H3 ligands, such as, for example, a histamine H3 receptor antagonist or inverse agonist may increase the release of these neurotransmitters in the brain. This suggests that histamine H3 receptor inverse agonists and antagonists could be used to improve cognitive deficits associated with neurodegenerative disorders such as AD.

Such combination products employ the compound of this invention within the dosage range described herein and the other pharmaceutically active compound or compounds within approved dosage ranges and/or the dosage described in the publication reference.

### Methods of Preparation

Another aspect of the present invention provides a process for preparing a compound of formula (I), or a pharmaceutically acceptable salt thereof, which process (wherein R¹ is, unless otherwise specified, as defined in formula (I)) comprises of:

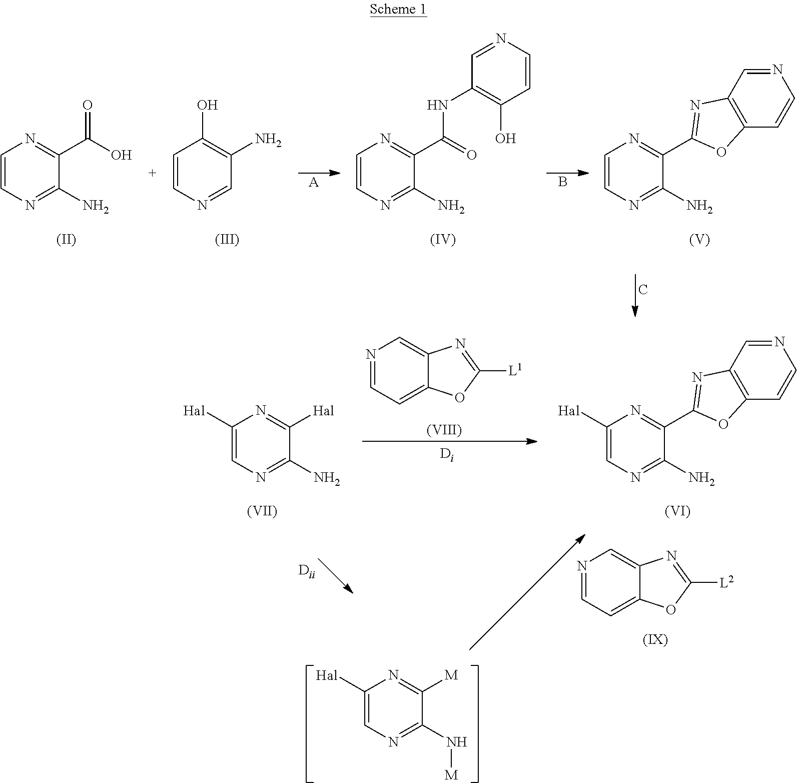

A. A compound of formula (II) may be reacted with a compound of formula (III) to give a compound of formula (IV) in the presence of an amide coupling reagent such as TBTU, HBTU, EDCI, CDI or TSTU in an organic solvent such as acetonitrile, DMF, dichloromethane or mixtures thereof, optionally in the presence of an additive such as HOBt or DMAP and an organic base such as triethylamine, DIPEA, pyridine or DMAP is added.
B. A compound of formula (IV) may be transformed into a compound of formula (V) in the presence of polyphosphoric acid or phosphoroxychloride optionally in the presence of water at temperatures in the range of about +100 to 220° C. or using triphenylphosphine in combination with a chlorinating agent such as hexachloroethane or tetrachloromethane and an organic base such as triethylamine in an organic solvent such as dichloromethane or dichloroethane at temperatures in the range of about −10 to +50° C.
C. A compound of formula (V) may be halogenated using a brominating agent such as NBS or bromine or an iodinating agent such as iodine or NIS in an organic solvent such as DMF, dichloromethane, methanol or acetic acid or mixtures thereof, at temperatures in the range of about 0 to +100° C. to obtain a compound of formula (VI).
D. i) A compound of formula (VII) may be reacted with a compound of formula (VIII), where L¹ is hydrogen or a metal such as lithium, magnesium, zinc or copper to obtain a compound of formula (VI). The reaction is performed in the presence of a metallic catalyst such as palladium(II), palladium(0), nickel(0), nickel(II) or cobalt(II), for example bis(triphenylphosphine)palladium(II) chloride, diacetoxypalladium(II), trisdibenzylideneacetone(dipalladium)(0), optionally in the presence of a phosphine ligand such as tricyclohexylphosphine or tri-n-butylphosphine. The reaction may be performed in the presence of an inorganic base such as potassium phosphate, sodium tert-butoxide, caesium carbonate, potassium carbonate or sodium carbonate, or
   ii) A compound of formula (VII) may be treated with an alkylmetal reagent such as BuLi, or an alkylmagnesium halide such as isopropylmagnesiumchloride lithium chloride complex to give an intermediate, where M is Li, MgCl lithium complex or MgBr lithium complex, which will further react with a compound of formula (IX), where L² is an appropriate leaving group such as chloride, thiomethyl, thiophenyl or triflate to obtain a compound of formula (VI). The reaction may be performed in ethereal solvents such as THF, diethyl ether or 1,4-dioxane, optionally in the presence of an additive such as sodium or potassium tert-butoxide, TMDEA or LiCl, at temperatures between about −100 and +50° C.

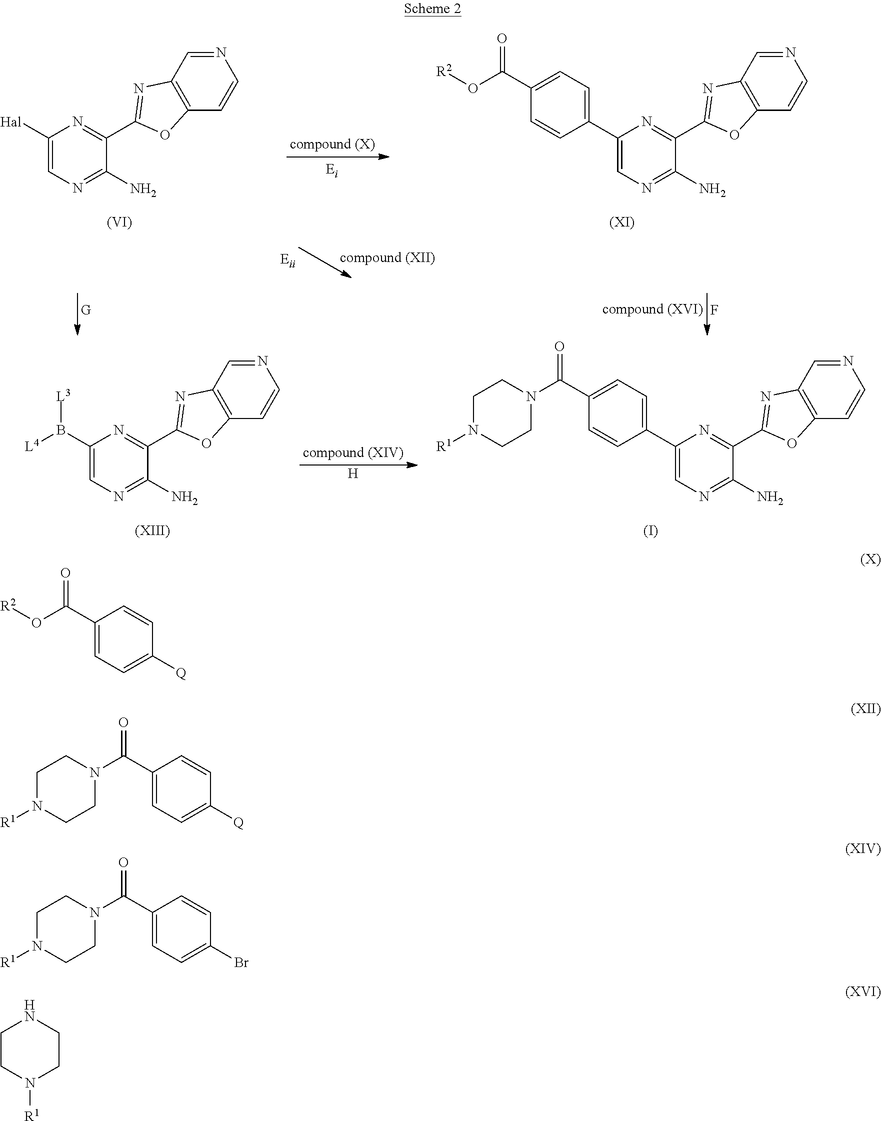

E. i) A compound of formula (VI) may be reacted with a compound of formula (X) using metal catalyzed cross-coupling reactions such as Suzuki, Negishi or Stille reactions to obtain a compound of formula (XI). Compound of formula (X) is defined by Q is B(L³)(L⁴), Sn(L⁵)₃, ZnCl, MgCl or MgBr, where L³ and L⁴ are ligands suitable for the boron atom such as hydroxyl or alkoxy, for example isopropoxy or L³ and L⁴ may be linked together to form for example 1, 3,2-dioxaborolane or 4,4,5,5-tetramethyl[1,3,2]dioxaborolane, L⁵ is a suitable ligand for the tin atom such as an alkyl group e.g. methyl or butyl, R² is a suitable C₁₋₄alkyl group. A suitable catalyst for the cross coupling reaction includes a transition metal catalyst such as palladium(0), palladium(II), nickel(0) or nickel(II) complexes, for example tetrakis(triphenylphosphine)palladium(0), dichloro[1,1′-bis(diphenylphosphino)ferrocene]palladium(II); dichloro[1,1′-bis(di-tert-butylphosphino)ferrocene]palladium(II) or bis(triphenylphosphine)palladium(II) chloride, in a suitable solvent such as water, ethanol, DMF, 1,4-dioxane, THF, IPA, toluene or mixtures thereof, at temperatures in the range about +10 to 200° C., for example in the range about +60 to 160° C., or in the range about +25 to 120° C., or in the range about +50 to 100° C. The reaction is conducted in the presence of a suitable base such as sodium, potassium or caesium carbonate, potassium or sodium acetate, or triethylamine, or
ii) A compound of formula (VI) may be reacted with a compound of formula (XII), wherein R¹ is hydrogen or methyl and Q is B(L³)(L⁴), Sn(L⁵)₃, ZnCl, MgCl or MgBr, where L³ and L⁴ are ligands suitable for the boron atom such as hydroxyl or alkoxy, for example isopropoxy or L³ and L⁴ may be linked together to form for example 1, 3,2-dioxaborolane or 4,4,5,5-tetramethyl[1,3,2]dioxaborolane, L⁵ is a suitable ligand for the tin atom such as an alkyl group e.g. methyl or butyl, to give a compound of formula (I) using the methods described in Eᵢ.
F. A compound of formula (XI), where R² is C₁₋₄alkyl may be transformed into a compound of formula (XI), where R² is hydrogen by hydrolysis under basic conditions using an inorganic base such as lithium, sodium or potassium hydroxide or under acidic conditions using an acid such as hydrochloric acid or sulphuric acid. The reaction may be performed in a suitable solvent such as water, THF or 1,4-dioxane or mixtures thereof. A compound of formula (XI), where R² is hydrogen may be transformed into a compound of formula (I) by reaction with a suitable amine of formula (XVI) in the presence of a suitable coupling reagent such as TBTU, HBTU, TSTU, CDI or EDCI and a base such as DIPEA, triethylamine or pyridine, optionally in the presence of an additive such as HOBt or DMAP. Alternatively, a compound of formula (XI), where R² is hydrogen is treated with a chlorinating agent such as oxalyl chloride or thionyl chloride, optionally in the presence of DMF at temperatures in the range of about 0 to +80° followed by reaction with a suitable amine compound of formula (XVI) in the presence of a suitable base such as pyridine, triethylamine or DIPEA in a suitable solvent such as dichloromethane, DMF, THF or acetonitrile to obtain a compound of formula (I).
G. A compound of formula (VI) may be converted into a compound of formula (XIII), where L³ and L⁴ are linked together to form for example 1, 3,2-dioxaborolane or 4,4,5,5-tetramethyl[1,3,2]dioxaborolane by reaction with a boron reagent such as bis(pinacolato)diboron or dioxaborolane in the presence of a suitable palladium catalyst such as 1′-bis(diphenylphosphino)ferrocene-palladium(II) dichloride or bis(triphenylphosphine)palladium(II) chloride in an organic solvent such as ethanol, DMF, 1,4-dioxane, THF or toluene at temperatures in the range about +10 to 200° C., for example in the range about +20 to 160° C. The reaction may be conducted in the presence of a suitable base such as sodium, potassium or caesium carbonate, potassium or sodium acetate, or triethylamine. Alternatively, a compound of formula (VI) may be converted into a compound (XIII), where L³ and L⁴ are alkoxy by lithiation with a suitable lithiating agent such as BuLi in an ethereal solvent such as diethylether, THF or 1,4-dioxane and then further reacted with an alkoxyborate, e.g. triisopropylborate. The reaction may be performed at temperatures in the range of about −100 to 0° C. A compound of formula (XIII), where L³ and L⁴ are alkoxy may be transformed into a compound of formula (XIII), where L³ and L⁴ are hydroxyl by hydrolysis in acidic media, for example hydrochloric or sulphuric acid.
H. A compound of formula (XIII) may be reacted with a compound of formula (XIV) to obtain a compound of formula (I) in the presence of a palladium catalyst such as 1′-bis(diphenylphosphino)ferrocene-palladium(II) dichloride, bistriphenylphosphine)palladium(II) chloride, trisdibenzylideneacetone(dipalladium), tetrakis(triphenylphosphine)palladium(0) in a suitable solvent such as water, ethanol, DMF, 1,4-dioxane, THF or toluene or mixtures thereof at temperatures in the range about +10 to 200° C., for example in the range about +60 to 160° C. The reaction is conducted in the presence of a suitable base such as sodium, potassium or caesium carbonate, potassium or sodium acetate, or triethylamine.

The obtained compound of formula (I) may be purified by a followed purification step, if necessary.

Crude compound of formula (I) may be purified by adding activated charcoal to an acidic aqueous solution of compound (1), preferably using hydrochloric acid. The charcoal can be removed by filtration for example through a plug of diatomeous earth. A water miscible organic solvent such as IPA may be added. An inorganic base such as sodium and potassium hydroxide is added to basic pH and precipitated compound (1) may be isolated by filtration.

Alternatively the compound may be purified by column chromatography on silica eluting with a suitable organic solvent or a mixture of solvents, preferably mixtures of dichloromethane and methanol optionally containing ammonia in methanol.

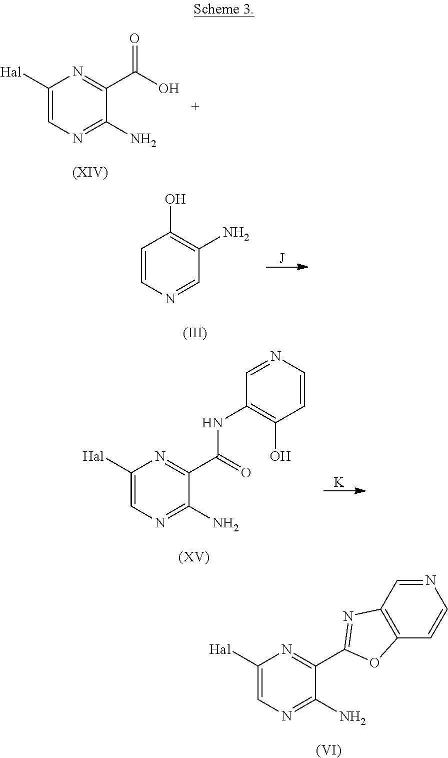

J. A compound of formula (XIV) may be reacted with a compound of formula (III) in the presence of a coupling reagent T₃P in the presence of an organic base, for example triethyl amine or Hunigs base to give a compound (XV). The reaction may be run in an organic solvent such as THF, DMF or acetonitrile at temperatures between about +20 to 80° C.
K. A compound of formula (XV) may be transformed into a compound of formula (VI using triphenylphosphine in combination with hexachloroethane and an organic base such as triethylamine in an organic solvent such as dichloromethane, dichloroethane, THF or 2-methylTHF at temperatures in the range of about −10 to +50° C.

It is understood by a person skilled in the art that the transformations described above might be used in a different order.

The compounds of the invention and intermediates may be isolated from their reaction mixtures, and if necessary further purified, by standard methods.

Compounds (II), (III) and (VII) are commercially available. Compounds (VIII), (IX), (X), (XII), (XIV) are commercially available or can be synthesised by a person skilled in the art using methods described in the literature (Heuser, S, Tetrahedron Lett, 2005, 46, 9001).

Further aspects of the invention are the products obtainable by the processes and or specific Examples disclosed herein.

### BRIEF DESCRIPTION OF DRAWINGS

FIG. 1 shows an X-ray powder diffractogram (XRDP) pattern for Example 13 (crystalline Form A).

FIG. 2 shows X-ray powder diffractogram (XRDP) pattern corresponding to Examples 8-12 (hydrates/dried hydrates).

I shows the XRDP for the anhydrate; Example 8.
II shows the XRDP for the hemihydrate; Example 9.
III shows the XRDP for the hydrate between hemi- and monohydrate; Example 10.
IV shows the XRDP for the monohydrate; Example 11.
V shows the XRDP for the dihydrate; Example 12.

### WORKING EXAMPLES

### Method 1

### 3-Amino-N-(4-hydroxypyridin-3-yl)pyrazine-2-carboxamide

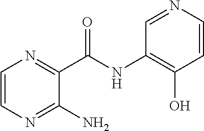

3-Amino-4-hydroxypyridine (14.29 g, 129.8 mmol) and 3-aminopyrazine-2-carboxylic acid (18.05 g, 130 mmol) were mixed in DMF (350 mL). Triethylamine (51 mL, 364 mmol) and O-benzotriazol-1-yl-N,N,N′,N′-tetra-methyluronium tetrafluoroborate (50.0 g, 155.7 mmol) were added and the mixture was stirred at RT under argon atmosphere overnight. The mixture was combined with a reaction mixture run under the same conditions as above. The solid formed was isolated by filtration and was washed with two portions of dichloromethane. The solid was dried under vacuum to give the title compound (32.35 g, 140 mmol, 54%). There was a precipitate in the mother liquor. The solid was isolated by filtration. The solid was washed with dichloromethane and was dried under vacuum to give the title compound (1.27 g, 5.49 mmol, 2%). There was a precipitate in the mother liquor. The solid was isolated by filtration. The solid was washed with dichloromethane and was dried under vacuum to give the title compound (3.29 g, 14.2 mmol, 6%). There was a precipitate in the mother liquor. The solid was isolated by filtration. The solid was washed with dichloromethane and was dried under vacuum to give the title compound (7.02 g, 30.4 mmol, 11%).

Total yield of 3-amino-N-(4-hydroxypyridin-3-yl)pyrazine-2-carboxamide (43.9 g, 73%).

MS (ESI⁺) m/z 232 [M+H]⁺.

¹H NMR (400 MHz, DMSO-d₆) δ ppm 11.63 (br. s., 1H) 10.55 (s, 1H) 8.80 (d, 1H) 8.30 (d, 1H) 7.93 (d, 1H) 7.71 (dd, 1H) 7.61 (br. s., 2H) 6.29 (d, 1H).

### Method 2

### 3-(Oxazolo[4,5-c]pyridin-2-yl)pyrazin-2-amine

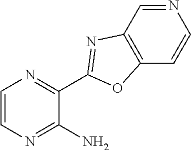

3-Amino-N-(4-hydroxypyridin-3-yl)pyrazine-2-carboxamide (17.3 g, 74.8 mmol), hexachloroethane (22.14 g, 93.5 mmol) and triphenylphosphine (29.4 g, 112 mmol) were mixed in dichloromethane (600 mL). The mixture was cooled on an ice-water bath. Triethylamine (42 mL, 300 mmol) was added and the mixture was stirred under argon for 5 min. The cooling bath was removed and the suspension was stirred at RT under argon atmosphere for 16 h. Hexachloroethane (11.07 g, 46.8 mmol) and triphenylphosphine (15.70 g, 59.9 mmol) were dissolved in dichloromethane (100 mL) in a separate flask. Triethylamine (21 mL, 150 mmol) was added. The mixture was added to the reaction mixture. The mixture was stirred at RT for 20 min. The solid was isolated by filtration and was washed with dichloromethane. The solid was slurried in water (400 ml) and was stirred for 20 min. The solid was isolated by filtration and was dried under vacuum to give the title compound (12.83 g, 60.2 mmol, 80%). The mother liquor from the reaction mixture was extracted with hydrochloric acid (aq, 1M, 200+400 ml). The combined aqueous phases were washed with dichloromethane (2×100 ml). NaHCO₃ (aq, sat) was added until neutral pH was obtained. The solid formed was isolated by filtration. The solid was washed with one portion of water (20 ml) and was dried under vacuum at 40° to give the title compound (1.68 g, 7.88 mmol, 11%).

Total yield: 3-(oxazolo[4,5-c]pyridin-2-yl)pyrazin-2-amine (14.51 g, 91%). For NMR analysis, 2 mg of the title compound was dissolved in DCl (1M in D₂O, 0.1 ml) and the sample was diluted with D₂O (0.7 ml).

MS (ESI⁺) m/z 214 [M+H]⁺.

¹H NMR (500 MHz, D₂O/DCl) δ ppm 9.37 (s, 1H) 8.79 (d, 1H) 8.34 (d, 1H) 8.23 (d, 1H) 8.07 (d, 1H).

### Method 3

### a) 5-Bromo-3-(oxazolo[4,5-c]pyridin-2-yl)pyrazin-2-amine

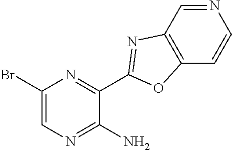

3-(Oxazolo[4,5-c]pyridin-2-yl)pyrazin-2-amine (23.51 g, 110.3 mmol) was slurried in acetic acid (500 mL). N-Bromosuccinimide (37.0 g, 208 mmol) was added and the mixture was stirred at RT overnight. Acetic acid (100 mL) was added followed by N-bromosuccinimide (9.81 g, 55.1 mmol) and the mixture was stirred vigorously for 1 h. N-Bromosuccinimide (5.0 g, 28.1 mmol) was added and the mixture was stirred for 2 h. The solid was isolated by filtration. The solid was stirred in water (400 ml) for 10 min. The solid was isolated by filtration. The solid was dried under vacuum at 50° overnight to give the title compound (23.1 g, 79 mmol, 72%)

The acetic acid filtrate was concentrated by evaporation. NaHCO₃ (sat, aq) was added under stirring until neutral pH was obtained. The solid formed was isolated by filtration and was washed with Na₂S₂O₃ (aq) followed by water. The solid was dried under vacuum at 50° overnight to give the title compound (5.28 g, 18.1 mmol, 16%). Solids in the combined mother liquors were isolated by filtration and were washed with water. The solid was dried under vacuum to give the title compound (2.25 g, 7.70 mmol, 7%). Total yield of 5-bromo-3-(oxazolo[4,5-c]pyridin-2-yl)pyrazin-2-amine: (30.6 g, 95%).

MS (ESI⁺) m/z 294 [M+H]⁺.

¹H NMR (500 MHz, DMSO-d₆) δ ppm 9.20 (s, 1H) 8.66 (d, 1H) 8.48 (s, 1H) 8.07 (br. s., 2H) 7.99 (d, 1H).

### b) An alternative method of preparing 5-Bromo-3-(oxazolo[4,5-c]pyridin-2-yl)pyrazin-2-amine

Dichloromethane (675.0 mL) was degassed through N₂ bubbling for 10 min. 6-Bromo-3-amino-N-(4-hydroxypyridin-3-yl)pyrazine-2-carboxamide (45.0 g, 0.131 mol) was added and the mixture was stirred for 15 min at 25° C. The mixture was cooled to 5° C. Triphenyl phosphine (78.82 g, 0.294 mol) was added followed by triethyl amine (100 ml, 0.719 mol). The mixture was stirred for 10 min at 5° C. A solution of hexachloroethane (56.34 g, 0.235 mol) in dichloromethane (225 mL) was prepared. 25-30% of the solution was added over a period of 25-30 min while maintaining the temperature at 5° C. The mixture was stirred for 30 min at 5° C. The remaining amount of hexachloroethane solution was added to the reaction mixture over a period of 1 h at 5° C. The temperature was raised to 15° C. and the mixture was stirred for 30 min. The product was isolated by filtration and the solid was washed with dichloromethane (180 mL) followed by acetonitrile (135 mL). The solid was slurried in water (675 mL) for 30 min and the solid was isolated by filtration. The solid was washed with water (225 mL) followed by acetonitrile (135 mL). The solid was dried under vacuum at 60° C. for 12 h to give the title compound (89%).

¹HNMR (400 MHz, DMSO-d₆): δ ppm 9.20 (s, 1H) 8.66 (d, 1H) 8.48 (s, 1H) 8.07 (br. s., 2H) 7.99 (d, 1H).

MS (ESI⁺): m/z 294 [M+H]⁺

### Method 4

### 6-Bromo-3-amino-N-(4-hydroxypyridin-3-yl)pyrazine-2-carboxamide

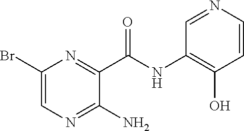

3-Amino-6-bromopyrazine-2-carboxylic acid (53.59 g, 0.229 mol) and 4-hydroxy-3-aminopyridine (33.64 g, 0.275 mol) were mixed in THF (500 mL) and triethyl amine (115.86 g, 1.145 mol) was added. The mixture was stirred at 25° for 10 min. The temperature was raised to 55° C. and T₃P (218.90 g, 0.344 mol) was added. The mixture was stirred at 55° C. for 30 min. Water (150 mL) was added at 55° C. and the mixture was stirred for 2 h at 55° C. Solvents were distilled of until fractions at 55-80° C. was removed.

Acetonitrile (500 mL) was added followed by water (350 mL) while maintaining the temperature at 75° C. The mixture was stirred for 2 h at 75° C. The solid was isolated by filtration. The solid was washed with water (250 mL) followed by acetonitrile (250 mL). The solid was dried under vacuum at 80° C. for 5 h to give the title compound (84.5%)

¹HNMR (400 MHz, DMSO-d₆): 11.64 (s, 1H) 10.26 (s, 1H) 8.78 (s, 1H) 8.45 (s, 1H) 7.78 (s, 2H) 7.72 (d, 1H) 6.31 (d, 1H).

MS (ESI⁺) m/z 310 [M+H]⁺

### Method 5

### tert-Butyl 4-(4-(5-amino-6-(oxazolo[4,5-c]pyridin-2-yl)pyrazin-2-yl)benzoyl)piperazine-1-carboxylate

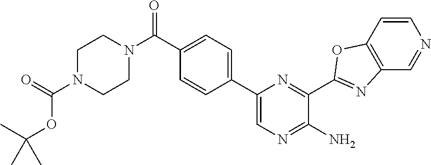

tert-Butyl 4-(4-(4,4,5,5-tetramethyl-1,3,2-dioxaborolan-2-yl)benzoyl)piperazine-1-carboxylate (0.314 g, 0.75 mmol), 5-bromo-3-(oxazolo[4,5-c]pyridin-2-yl)pyrazin-2-amine (0.200 g, 0.68 mmol) and PdCl₂(dppf)-CH₂Cl₂Adduct (0.028 g, 0.03 mmol) were mixed in THF (4 mL). Sodium carbonate (2M, aq) (1.0 mL, 2.0 mmol) was added. Argon was bubbled through the mixture for 1 min. The mixture was heated at 130° C. for 40 min. in a microwave reactor. The residue was evaporated onto silica. The mixture was purified by column chromatography on silica eluting with gradients of methanol and ammonia in dichloromethane. The fractions containing product were evaporated to give the title compound as a solid (0.197 g, 57%).

¹H NMR (500 MHz, CDCl₃) δ ppm 9.19 (s, 1H) 8.76 (s, 1H) 8.70 (d, 1H) 8.09 (m, 2H) 7.74 (d, 1H) 7.57 (m, 2H) 3.36-3.89 (m, 8H) 1.49 (s, 9H).

MS (APCI⁺) m/z 502 (M+H)⁺.

### Example 1

### First Method

### (4-(5-Amino-6-(oxazolo[4,5-c]pyridin-2-yl)pyrazin-2-yl)phenyl)(4-methylpiperazin-1-yl)methanone

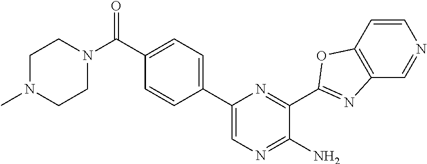

(4-Methylpiperazin-1-yl)(4-(4,4,5,5-tetramethyl-1,3,2-dioxaborolan-2-yl)phenyl)methanone (187 mg, 0.56 mmol) and 5-bromo-3-(oxazolo[4,5-c]pyridin-2-yl)pyrazin-2-amine (150 mg, 0.51 mmol; obtained by Method 3) were charged to a MW-vial and THF (3 mL) was added followed by sodium carbonate (2M, aq) (0.770 mL, 1.54 mmol). Argon was bubbled through the mixture for 2 min. PdCl₂(dppf)-CH₂Cl₂ adduct (29 mg, 0.04 mmol) was added and the mixture was heated at 130° C. for 40 min. The mixture was mixed with the reaction mixture from a reaction performed as above. The mixture was diluted with dichloromethane and methanol. Diatomeous earth was added and the solvents were evaporated. The diatomeous earth was loaded in a pre-column and the mixture was purified by column chromatography on silica eluting with gradients of methanol and ammonia in dichloromethane. The fractions containing product were pooled and evaporated to give the title compound as a solid (0.37 g, 87%).

¹H NMR (500 MHz, DMSO-d₆) δ ppm 9.22 (s, 1H) 9.02 (s, 1H) 8.67 (d, 1H) 8.17 (m, 2H) 8.10 (br. s., 2H) 8.04 (dd, 1H) 7.54 (m, 2H) 3.64 (br. s., 2H) 3.38 (br. s., 2H) 2.36 (br. s., 2H) 2.30 (br. s., 2H) 2.21 (s, 3H).

### Example 1

### Second Method

### (4-(5-Amino-6-(oxazolo[4,5-c]pyridin-2-yl)pyrazin-2-yl)phenyl)(4-methylpiperazin-1-yl)methanone

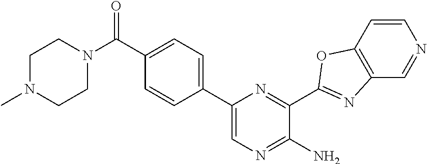

(4-Methylpiperazin-1-yl)(4-(4,4,5,5-tetramethyl-1,3,2-dioxaborolan-2-yl)phenyl)methanone (137 g, 413.50 mmol), 5-bromo-3-(oxazolo[4,5-c]pyridin-2-yl)pyrazin-2-amine (152.5 g, 375.91 mmol), potassium carbonate (2M, aq) (564 mL, 1127.73 mmol) and PdCl₂(dppf)-CH₂Cl₂ adduct (15.35 g, 18.80 mmol) were mixed in DMF (430 mL) under argon atmosphere. The mixture was heated at 100° C. for 2.5 h. Charcoal (˜75 mL) was added to the reaction mixture at 100° C. the mixture was cooled to RT and then vacuum filtered through a short pad of diatomeous earth. The first fraction containing DMF-water was discarded. The plug was washed with methanol followed by dichloromethane and methanol.

The filtrate was evaporated and the residue was purified on a short silica column (1.8 kg silica) eluting with dichloromethane:methanol (5:1). The pure fractions were combined and evaporated to give a solid (143 g, 92%). The solid was combined with material prepared using the method described above (128 g and 39 g). The solid was stirred in water (4.0 L) at pH 8 (NaOH solid added to pH 8) for 48 h. The solid was isolated by filtration and was dried under vacuum at 40° C. overnight to give the title compound (297 g).

¹H NMR (500 MHz, DMSO-d₆) δ ppm 9.22 (d, 1H) 9.01 (s, 1H) 8.66 (d, 1H) 8.16 (m, 2H) 8.09 (br. s., 2H) 8.03 (dd, 1H) 7.53 (m, 2H) 3.64 (br. s., 2H) 3.38 (br. s., 2H) 2.45-2.26 (m, 4H) 2.22 (s, 3H).

### Example 2

### (4-(5-Amino-6-(oxazolo[4,5-c]pyridin-2-yl)pyrazin-2-yl)phenyl)(4-methylpiperazin-1-yl)methanone hydrochloride

(4-(5-Amino-6-(oxazolo[4,5-c]pyridin-2-yl)pyrazin-2-yl)phenyl)(4-methylpiperazin-1-yl)methanone (25 mg, 0.06 mmol) was dissolved in methanol and dichloromethane (20 ml). HCl (1M in diethyl ether, 1 ml) was added. The solid was isolated by filtration and was dried under vacuum at 50° C. for 1.5 h. to give the title compound as the hydrochloride salt (0.299 g, 65%).

MS (ESI⁺) m/z 416[M+H]⁺.

The sample for NMR analysis was dissolved in DCl (1 M in D₂O, 0.1 ml) and was diluted with D₂O (0.7 ml) ¹H NMR (500 MHz, D₂O, DCl) δ ppm 9.08 (s, 1H) 8.61 (d, 1H) 8.54 (s, 1H) 8.02 (d, 1H) 7.80-7.88 (m, 2H) 7.42-7.50 (m, 2H) 3.64 (br. s., 1H) 3.55 (br. s., 1H) 3.46 (br. s., 1H) 3.32 (br. s., 1H) 3.17 (br. s., 2H) 2.92 (s, 3H).

### Example 3

### (4-(5-Amino-6-(oxazolo[4,5-c]pyridin-2-yl)pyrazin-2-yl)phenyl)(4-methylpiperazin-1-yl)methanone mesylate

(4-(5-Amino-6-(oxazolo[4,5-c]pyridin-2-yl)pyrazin-2-yl)phenyl)(4-methylpiperazin-1-yl)methanone (25 mg, 0.06 mmol) was dissolved in dichloromethane (1.875 mL) and methanol (0.375 mL) and methanesulfonic acid in methanol (1 M, 0.060 mL, 0.06 mmol) were added. The resulting solution was concentrated to dryness by careful stirring over night. The residue was suspended in dichloromethane (1 mL) and the solid was collected by filtration. The solid was dried at 50° C. under vacuum for 16 h to give the title compound (25 mg, 81%).

¹H NMR (500 MHz, D₂O) δ ppm 9.35 (s, 1H) 8.78 (dd, 1H) 8.71 (s, 1H) 8.31 (d, 1H) 7.97 (m, 2H) 7.52 (m, 2H) 3.97 (d, 1H) 3.65-3.05 (m, 6H) 2.87 (s, 3H) 2.68 (s, 3H)

MS (APCI⁺) m/z 416[M+H]⁺.

### Example 4

### (4-(5-Amino-6-(oxazolo[4,5-c]pyridin-2-yl)pyrazin-2-yl)phenyl)(4-methylpiperazin-1-yl)methanone sulfate

(4-(5-Amino-6-(oxazolo[4,5-c]pyridin-2-yl)pyrazin-2-yl)phenyl)(4-methylpiperazin-1-yl)methanone (25 mg, 0.06 mmol) was dissolved in dichloromethane (1.875 mL) and methanol (0.375 mL) and sulfuric acid in methanol (1M, 0.060 mL, 0.06 mmol) was added. The resulting suspension was concentrated to dryness by careful stirring over night. The residue was suspended in dichloromethane (1 mL) and the solid was collected by filtration. The solid was dried at 50° C. under vacuum for 16 h to give the title compound (24 mg, 77%).

¹H NMR (500 MHz, D₂O) δ ppm 9.35 (s, 1H) 8.78 (d, 1H) 8.71 (s, 1H) 8.31 (d, 1H) 7.98 (m, 2H) 7.52 (m, 2H) 3.98 (d, 1H) 3.65-3.08 (m, 6H) 2.87 (s, 3H)

MS (APCI⁺) m/z 416[M+H]⁺.

### Example 5

### (4-(5-Amino-6-(oxazolo[4,5-c]pyridin-2-yl)pyrazin-2-yl)phenyl)(4-methylpiperazin-1-yl)methanone maleate

(4-(5-Amino-6-(oxazolo[4,5-c]pyridin-2-yl)pyrazin-2-yl)phenyl)(4-methylpiperazin-1-yl)methanone (25 mg, 0.06 mmol) was dissolved in dichloromethane (1.875 mL) and methanol (0.375 mL) and maleic acid in MeOH (1 M, 0.060 mL, 0.06 mmol) was added. The resulting solution was concentrated to dryness by careful stirring over night. The residue was suspended in dichloromethane and the solid was collected by filtration. The solid was dried at 50° C. under vacuum for 16 h to give the title compound (26 mg, 80%).

¹H NMR (500 MHz, D₂O) δ ppm 9.36 (s, 1H) 8.79 (d, 1H) 8.72 (s, 1H) 8.32 (d, 1H) 7.99 (m, 2H) 7.53 (m, 2H) 6.33 (s, 2H) 3.98 (d, 1H) 3.65-3.07 (m, 6H) 2.87 (s, 3H)MS (APCI⁺) m/z 416[M+H]⁺.

### Example 6

### (4-(5-Amino-6-(oxazolo[4,5-c]pyridin-2-yl)pyrazin-2-yl)phenyl)(4-methylpiperazin-1-yl)methanone tosylate

(4-(5-Amino-6-(oxazolo[4,5-c]pyridin-2-yl)pyrazin-2-yl)phenyl)(4-methylpiperazin-1-yl)methanone (25 mg, 0.06 mmol) was dissolved in dichloromethane (1.875 mL) and methanol (0.375 mL) and p-toluenesulfonic acid monohydrate in MeOH (1M, 0.060 mL, 0.06 mmol) was added. The resulting solution was concentrated to dryness by careful stirring over night. The residue was suspended in dichloromethane. The solid was collected by filtration. The solid was dried at 50° C. under vacuum for 16 h to give the title compound (12 mg, 33%).

¹H NMR (500 MHz, D₂O) δ ppm 9.35 (s, 1H) 8.78 (d, 1H) 8.71 (s, 1H) 8.31 (d, 1H) 7.98 (d, 2H) 7.52 (d, 2H) 7.56 (d, 2H) 7.24 (d, 2H) 4.64 (d, 2H) 3.98 (d, 1H) 3.65-3.07 (m, 6H) 2.87 (s, 3H) 2.27 (s, 3H)

MS (APCI⁺) m/z 416[M+H]⁺.

### Example 7

### (4-(5-Amino-6-(oxazolo[4,5-c]pyridin-2-yl)pyrazin-2-yl)phenyl)(4-methylpiperazin-1-yl)methanone hydrobromide

(4-(5-Amino-6-(oxazolo[4,5-c]pyridin-2-yl)pyrazin-2-yl)phenyl)(4-methylpiperazin-1-yl)methanone (100 mg, 0.24 mmol) was dissolved in dichloromethane (7.5 mL) and methanol (1.5 mL) (some heating required) and HBr (1M in H2O/MeOH) (0.241 mL, 0.24 mmol) was added. The resulting cloudy solution was concentrated to ˜5 mL and the resulting precipitate was collected by filtration. The solid was dried in vacuum at 50° C. for 16 h to give the title compound (101 mg, 73%).

¹H NMR (500 MHz, D₂O) δ ppm 9.29 (s, 1H) 8.73 (d, 1H) 8.66 (s, 1H) 8.26 (d, 1H) 7.92 (m, 2H) 7.47 (m, 2H) 3.94 (d, 1H) 3.65-3.03 (d, 6H) 2.84 (s, 3H)

MS (APCI⁺) m/z 416[M+H]⁺.

### Example 8

### (4-(5-Amino-6-(oxazolo[4,5-c]pyridin-2-yl)pyrazin-2-yl)phenyl)(4-methylpiperazin-1-yl)methanone anhydrate

(4-(5-Amino-6-(oxazolo[4,5-c]pyridin-2-yl)pyrazin-2-yl)phenyl)(4-methylpiperazin-1-yl)methanone (obtained in Example 1, second method; circa 10 mg) was dried under a nitrogen gas stream at 25° C. with a relative humidity of 0% RH or of 5% RH giving the anhydrate.

### Example 9

### (4-(5-Amino-6-(oxazolo[4,5-c]pyridin-2-yl)pyrazin-2-yl)phenyl)(4-methylpiperazin-1-yl)methanone hemihydrate

(4-(5-Amino-6-(oxazolo[4,5-c]pyridin-2-yl)pyrazin-2-yl)phenyl)(4-methylpiperazin-1-yl)methanone obtained in Example 8 was humidified under the same conditions as the anhydrate above but at 20% RH.

### Example 10

### (4-(5-Amino-6-(oxazolo[4,5-c]pyridin-2-yl)pyrazin-2-yl)phenyl)(4-methylpiperazin-1-yl)methanone intermediate hydrate between hemi- and monohydrate

The hemihydrate obtained in Example 9 was further humidified under the same conditions as the anhydrate but at a relative humidity between 20 and 60% RH such as 30% RH.

### Example 11

### (4-(5-Amino-6-(oxazolo[4,5-c]pyridin-2-yl)pyrazin-2-yl)phenyl)(4-methylpiperazin-1-yl)methanone monohydrate

The monohydrate was formed from an intermediate hydrate of obtained in Example 10 under the same conditions but at a relative humidity of 60% RH.

### Example 12

### (4-(5-Amino-6-(oxazolo[4,5-c]pyridin-2-yl)pyrazin-2-yl)phenyl)(4-methylpiperazin-1-yl)methanone dihydrate

The dihydrate was formed from the monohydrate obtained in Example 11 under the same conditions but at a relative humidity of 90% RH.

Examples 8-12 describe a group of crystal modifications, different from Form A, consisting of an anhydrate, a hemihydrate, a monohydrate, a dihydrate and intermediate forms between hemi and monohydrate. In this document, we use “hydrates/dried hydrates” as a common name for those. Conversion between those hydrates/dried hydrates occurs in air, depending on relative humidity.

Example XRPD patterns are shown in FIG. 2. The peak at about 25.5° 2Theta emanates from NIST SRM 676 alumina (α-Al₂O₃) standard material. Two diffractions, here called “Peak 1” and “Peak 2”, differ slightly due to state of hydration. “Peak 1” between 6.00 and 5.75° 2θ and Peak 2 between 11.96 and 11.46° 2θ. Peak 2 is a first multiple of Peak 1. The diffractions as measured for each state of hydration are (for Peak 1 and Peak 2), for anhydrate: 6.00 and 11.96° 2θ, for hemihydrate: 5.87 and 11.69° 2θ, for monohydrate: 5.82 and 11.60° 2θ and for dihydrate: 5.75 and 11.46° 2θ.

It will be appreciated by a person skilled in the art that the XRPD intensities may vary between different samples and different sample preparations for a variety of reasons including preferred orientation. It will also be appreciated by a person skilled in the art that smaller shifts in the measured Angle and hence the d-spacing may occur for a variety of reasons including variation of sample surface level in the diffractometer.

A person skilled in the art will realize that transformation time from one modification to another can vary depending on factors such as particle size, porosity of the powder bed, temperature and gas flow.

A sample of the material from Example 1 (second method) was kept in an XRPD-sample chamber under the same humidity conditions as described in Examples 8 to 12 and analysed by XRPD. The XRPD pattern confirmed the solid materials to be crystalline.

X-Ray Powder Diffraction (XRPD) patterns were collected on a theta-theta system (X′Pert PRO MPD, PANalytical, the Netherlands) using long-fine-focus Cu Kα-radiation, wavelength of X-rays 1.5418 Å, at 45 kV and 40 mA. A programmable divergence slit and a programmable anti-scatter slit giving an irradiated length of 6 mm were used. 0.02 radian Soller slits were used on the incident and on the diffracted beam path. A 20 mm fixed mask was used on the incident beam path and a Nickel-filter was placed in front of a PIXcel-detector using 255 active channels. A thin flat sample was prepared together with a small amount of NIST SRM 676 alumina (α-Al₂O₃) standard on a flat zero background plate made of silicon using a spatula. The plate was mounted in a sample holder and held still in a horizontal position during measurement in a chamber for controlled humidity and temperature (CHC, Anton Paar, Austria) designed to be used in the diffractometer. A number of diffraction patterns were collected between 5° 2theta and 50° 2theta in a continuous scan mode at one hour-intervals. Total time for a scan between 5 and 50° 2theta was approximately 10 minutes. The relative humidity in the chamber was controlled using a relative humidity generator (VTI RH-200) programmed to keep the humidity at defined levels. The temperature in the chamber was approximately 25° C.

### Peak Search on XRDP Data of the Hydrates Obtained in Example 8-12.

Manual peak angle estimations were done within the X′Pert High Score Plus software version 2.0. No background withdrawal or K_{α2}-stripping was done. Angle correction against internal standard of α-Al₂O₃ was done.

### Hygroscopicity

### Description of the Dynamic Vapor Sorption (DVS) Method Used

The weight change over time as a function of relative humidity (RH) at 25° C. was measured in a DVS Advantage, equipped with a SMS ultrabalance using DVS Advantage Control Software Version 1.2.0.21, from Surface Measurement Systems, London, UK.

Carrier gas was nitrogen 200 ml/minute. The sample amount was circa 10 mg. The equilibrium criteria, for each step, was set to 0.002%/minute with DMDT Window 5 minutes, DMDT Min Time 10 minutes and DMDT Max Time 360 minutes as defined in the Control Software. DMDT uses the percentage rate of change of mass with time (dm/dt) to determine whether a sample has come to equilibrium.

DMDT Window (min): The time window, over which a straight line is fitted to the mass data in order to calculate a value of dm/dt.

DMDT Min Time (min): The time that the measured dm/dt must satisfy the specified dm/dt criteria before switching to the next Method Stage.

DMDT Max Time (min): The maximum time a Method Stage will run in dm/dt mode before switching to the next Method Stage—regardless of whether the dm/dt criterion has been satisfied.

The sample was initially dried at 0% RH and the water vapor sorption was measured as weight gain after stepwise increase to 10, 20, 30, 40, 50, 60, 70, 80, 90 and 95% relative humidity. After stepwise drying using the same relative humidities in reverse order down to 0% RH a second sorption curve was obtained. The results are shown in Table 1.

TABLE 1

| | | | | Weight gain |
|---|---|---|---|---|
| | Weight gain | | | calculated as |
| | first | Weight gain | | molar |
| Relative | sorption | second sorption | Average | equivalents of |
| humidity | curve | curve | weight gain | water |
| 0% RH | 0.00% | 0.00% | 0.0% | 0.0 |
| 10% RH | 0.12% | 0.14% | 0.1% | 0.0 |
| 20% RH | 2.34% | 2.41% | 2.4% | 0.5 |
| 30% RH | 3.09% | 3.14% | 3.1% | 0.7 |
| 60% RH | 4.72% | 4.65% | 4.7% | 1.1 |
| 90% RH | 9.16% | 9.19% | 9.2% | 2.1 |

It will be appreciated by a person skilled in the art that variations in sorption can occur due to impurities in the sample and sample properties such as particle size, porosity and degree of crystallinity. Hence, the values given are not to be seen as absolute values.

### Example 13

### (4-(5-Amino-6-(oxazolo[4,5-c]pyridin-2-yl)pyrazin-2-yl)phenyl)(4-methylpiperazin-1-yl)methanone Crystalline Form A

5-Bromo-3-(oxazolo[4,5-c]pyridin-2-yl)pyrazin-2-amine (obtained in Method 3b); 41.28 g, 0.141 mol) was slurried in 1,4-dioxane (420 mL). (4-Methylpiperazin-1-yl)(4-(4,4,5,5-tetramethyl-1,3,2-dioxaborolan-2-yl)phenyl)methanone (53.67 g, 0.162 mol), potassium carbonate (39.07 g, 0.282 mol) and PdCl₂(dppf)-CH₂Cl₂ adduct (Pd-106) (5.77 g, 0.007 mol) were added. Water (84 mL) was added and the mixture was stirred for 10 min. The mixture was stirred at 55° C. for 1.5 h. The mixture was let to 45° C. and water (840 mL) was added. The mixture was let to 23° C. and the mixture was stirred for 15 h at 23° C. The solid was isolated by filtration the solid was washed with water (210 mL) followed by isopropyl alcohol (210 mL). The solid was dried under vacuum at 60° C. for 12 h to give the crude title compound (82%).

The crude title compound (31.52 g, 0.075 mol) was stirred in water (320 mL) for 10 min. HCl (24.50 g, 0.235 mol) in water (20 mL) was added. A slurry of activated charcoal (10 g, 25% w/w) in water (80 mL) was added and the mixture was stirred for 2 h at 23° C. The mixture was filtered through a plug of diatomeous earth. The filter plug was washed with water (20 mL). Isopropyl alcohol (120 mL) was added to the filtrate. A solution of sodium hydroxide (9.80 g, 0.235 mol) in water (120 mL) was added. The mixture was stirred for 20-30 min and the precipitated solid was isolated by filtration. The solid was washed with water (400 mL) followed by isopropyl alcohol (120 mL). The solid was dried by suction of air through the filter cake for 20 min. The solid was stirred in isopropyl alcohol (360 mL) and water (40 mL) at 55° C. for 1 h. The mixture was cooled to 23° and the mixture was stirred for 1 h. The solid was isolated by filtration and washed with isopropyl alcohol (80 mL). The solid was dried under vacuum at 60° C. for 10-12 h to give the title compound (78%).

¹HNMR (400 MHz, DMSO-d₆): δ ppm 9.22 (s, 1H) 9.02 (s, 1H) 8.67 (d, 1H) 8.17 (m, 2H) 8.10 (br. s., 2H) 8.04 (dd, 1H) 7.54 (m, 2H) 3.64 (br. s., 4H) 2.34 (br. s., 4H) 2.21 (s, 3H).

MS [ESI⁺]: m/z 416[M+H]⁺.

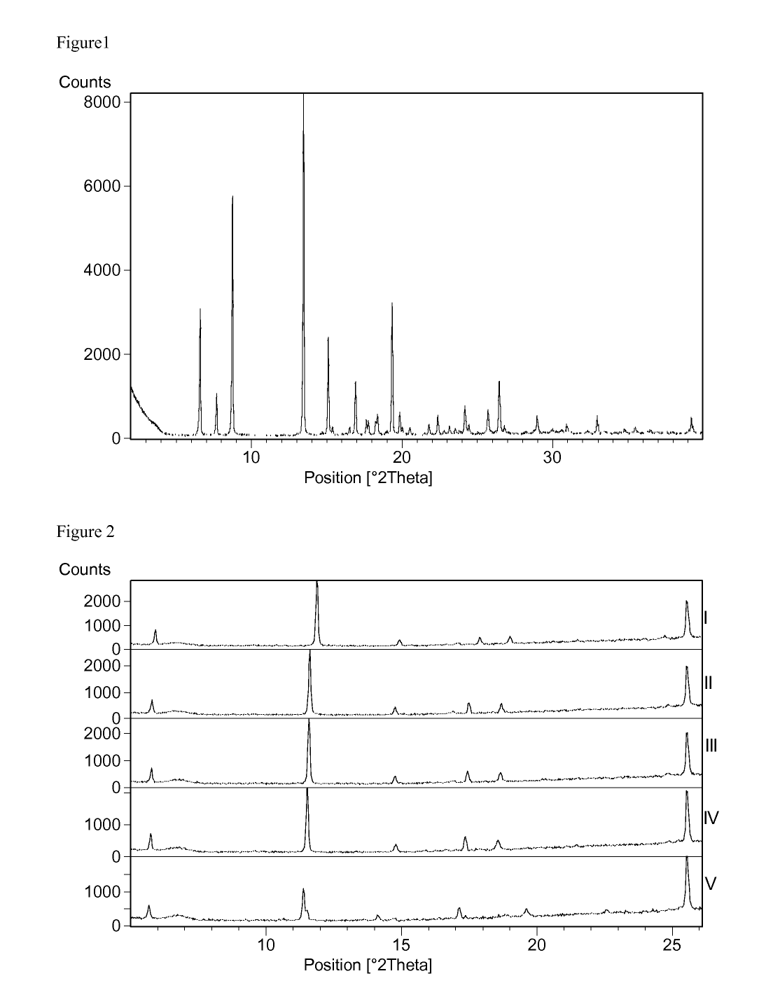

The solid product obtained was analysed by XRPD. A representative XRPD pattern is shown in FIG. 1. Selected peaks are provided in Table 2. The XRPD pattern confirmed the solid material to be Crystalline Form A.

TABLE 2
Selected XRPD peaks of Crystalline Form A

| | Measured | |
|---|---|---|
| | Angle | Relative |
| Peak | [°2Th.] | intensity |
| 1 | 6.58 | s |
| 2 | 7.67 | m |
| 3 | 8.73 | vs |
| 4 | 13.43 | vs |
| 5 | 15.08 | s |
| 6 | 16.49 | w |
| 7 | 16.89 | m |
| 8 | 17.61 | m |
| 9 | 17.74 | m |
| 10 | 19.31 | s |
| 11 | 19.82 | m |
| 12 | 22.35 | w |

An XRDP pattern for form A was collected using an XRPD instrumentation as described below.

X-Ray Powder Diffraction (XRPD) patterns were collected on a PANalytical X′Pert PRO MPD theta-theta system using long-fine-focus Cu Kα-radiation, wavelength of X-rays 1.5418 Å, at 45 kV and 40 mA. A programmable divergence slit and a programmable anti-scatter slit giving an irradiated length of 10 mm were used. 0.02 radian Soller slits were used on the incident and on the diffracted beam path. A 20 mm fixed mask was used on the incident beam path and a Nickel-filter was placed in front of a PIXcel-detector using 255 active channels. Thin flat samples were prepared on flat silicon zero background plates using a spatula. The plates were mounted in sample holders and rotated in a horizontal position during measurement. Diffraction patterns were collected between 2° 2theta and 40° 2theta in a continuous scan mode. Total time for a scan between 2 and 40° 2theta was approximately 10 minutes.

### Peak Search on XRDP Data of Crystalline Form A.

A manual peak search was done preceded by an angle correction against NIST SRM 676 alumina (α-Al₂O₃) standard.

The measured relative intensities vs. the strongest peak are given as very strong (vs) above 50%, as strong (s) between 25 and 50%, as medium (m) between 10 and 25%, as weak (w) between and 10% and as very weak (vw) under 5% relative peak height. It will be appreciated by a person skilled in the art that the XRPD intensities may vary between different samples and different sample preparations for a variety of reasons including preferred orientation. It will also be appreciated by a person skilled in the art that smaller shifts in the measured Angle and hence the d-spacing may occur for a variety of reasons including variation of sample surface level in the diffractometer.

### Example 14

### (4-(5-Amino-6-(oxazolo[4,5-c]pyridin-2-yl)pyrazin-2-yl)phenyl)(piperazin-1-yl)methanone

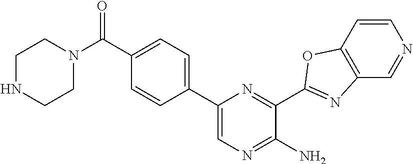

tert-Butyl 4-(4-(5-amino-6-(oxazolo[4,5-c]pyridin-2-yl)pyrazin-2-yl)benzoyl)piperazine-1-carboxylate (93 mg, 0.19 mmol; obtained by Method 5) was dissolved in dichloromethane (5 ml) and methanol (3 drops). Trifluoroacetic acid (1 ml, 12.98 mmol was added and the mixture was stirred at RT for 1 h. The mixture was concentrated by evaporation. Water (10 ml) was added. Na₂CO₃ (2M, aq) was added to basic pH. The mixture was extracted with dichloromethane (×3). The combined organic phases were dried and evaporated to give the title compound (77 mg, quant) as a solid. The solid was dissolved in dichloromethane (6 ml) and a few drops of methanol. HCl (1 M in diethylether, 0.20 ml) was added. The solid formed was isolated by filtration. The solid was washed with dichloromethane and was dried under vacuum at 50° C. to give (4-(5-amino-6-(oxazolo[4,5-c]pyridin-2-yl)pyrazin-2-yl)phenyl)(piperazin-1-yl)methanone (60 mg, 74%) as the hydrochloride salt.

¹H NMR (500 MHz, DMSO-d₆) δ ppm 9.41 (s, 1H) 9.27 (br. s., 2H) 9.08 (s, 1H) 8.81 (d, 1H) 8.27 (d, 1H) 8.20 (d, 2H) 8.14 (br. s., 2H) 7.63 (d, 2H) 3.18 (br. s., 4H).

MS (APCI⁺ m/z 402 (M+H)⁺.

### Example 15

### 4-(4-(5-Amino-6-(oxazolo[4,5-c]pyridin-2-yl)pyrazin-2-yl)benzoyl)-1-methylpiperazine 1-oxide

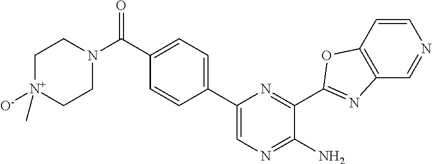

(4-(5-Amino-6-(oxazolo[4,5-c]pyridin-2-yl)pyrazin-2-yl)phenyl)(4-methylpiperazin-1-yl)methanone (Example 1; 43 mg, 0.10 mmol) was slurried in dichloromethane (2 mL). m-CPBA (24 mg, 0.10 mmol) was added and the mixture was stirred overnight. The residue was partitioned between NaHCO₃ (aq) and dichloromethane containing 5% of methanol. The aqueous phase was extracted with dichloromethane containing approx 5% methanol (×5). The organic phases were combined and the solvents were evaporated. The residue was dissolved in DMSO (0.5 ml), water (0.5 ml) and methanol (0.5 ml). The mixture was filtered and the mixture was purified by preparative HPLC using gradients of ammonium acetate (aq) and acetonitrile. The fractions containing product were pooled. The solvents were removed in a vacuum centrifuge. The solid was dried under vacuum at 50° C. to give 4-(4-(5-amino-6-(oxazolo[4,5-c]pyridin-2-yl)pyrazin-2-yl)benzoyl)-1-methylpiperazine 1-oxide (11 mg, 22%) as the acetic acid salt.

¹H NMR (500 MHz, CDCl₃) δ ppm 9.19 (s, 1H) 8.74 (s, 1H) 8.69 (d, 1H) 8.11 (m, 2H) 7.71 (d, 1H) 7.59 (m, 2H) 3.45 (s, 3H).

MS (APCI⁺) m/z 432 (M+H)⁺.

### Example 16

### {4-[5-amino-6-([1,3]oxazolo[4,5-c]pyridin-2-yl)pyrazin-2-yl]phenyl}(4-methylpiperazin-1-yl)[¹⁴C]methanone

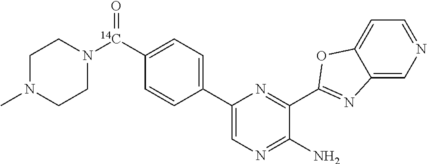

(4-Methylpiperazin-1-yl)(4-(4,4,5,5-tetramethyl-1,3,2-dioxaborolan-2-yl)phenyl) [¹⁴C]methanone, 5-bromo-3-(oxazolo[4,5-c]pyridin-2-yl)pyrazin-2-amine (330 mg, 0.90 mmol), PdCl₂(dppf) (33.0 mg, 0.05 mmol) and sodium carbonate (aq, 2M; 1.35 mL, 2.71 mmol) were mixed in tetrahydrofuran (3.5 mL). Argon was bubbled through the mixture. The mixture was heated in a microwave oven at 130° C. for 40 min. Water and ethyl acetate were added and the mixture was filtered though a disc frit. The mixture was extracted with dicholomethane (×5), dried (Na₂SO₄) and the solvents were evaporated. The residue was purified by column chromatography on silica eluting with gradients of methanol and dichloromethane. Fractions containing product were pooled and evaporated. The residue was triturated with ethanol to give the title compound (96 mg, 25.5%) as a solid with a specific radioactivity of 2.2 GBq/mmol. MS (ESI⁺) m/z 418 [M+H]+

### i) 4-Bromo(carboxy-¹⁴C)benzoic acid

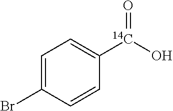

1,4-Dibromobenzene (472 mg, 2.00 mmol) dissolved in tetrahydrofuran (8 mL) was cooled at −78° C. n-Butyl lithium (1.251 mL, 2.00 mmol) was added slowly and the reaction was stirred at −78° C. for 1 h. The reaction was exposed to [¹⁴C]carbon dioxide (92 mg, 2.00 mmol) and was stirred for 1 h at ambient temperature. Water was added and the mixture was washed with diethyl ether (×3). HCl (conc.) was added to acidic pH. The mixture was extracted with diethyl ether (×3) and dried (Na₂SO₄). The solvents were evaporated to give the title compound (299 mg, 1.47 mmol, 73%) that was used in the next step without further purification.

### ii) (4-Bromophenyl)(4-methylpiperazin-1-yl)[¹⁴C]methanone

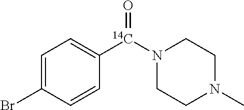

Thionyl chloride (1.075 mL, 14.73 mmol) and DMF (two drops) were added to a slurry of 4-bromo(carboxy-¹⁴C)benzoic acid (299 mg, 1.47 mmol) in toluene (8 mL). The mixture was stirred at 70° C. for 1 h. The solvent was evaporated by a stream of nitrogen and gentle heating. Dichloromethane (2 ml) was added and was evaporated by a stream of nitrogen. The residue was dissolved in dichloromethane (8 mL) and 1-methylpiperazine (0.197 mL, 1.77 mmol) dissolved in dichloromethane (1 mL) was added dropwise. The mixture was stirred for 1 h. NaOH (aq, 2M, 2 mL) was added. The organic layer was separated and dried (Na₂SO₄). The residue was purified by column chromatography on silica eluting with dichloromethane: methanol:7 N ammonia in methanol 100:5:1 Fractions containing product were pooled and the solvents were evaporated to give the title compound (269 mg, 0.94 mmol, 64%).

### iii) (4-Methylpiperazin-1-yl)(4-(4,4,5,5-tetramethyl-1,3,2-dioxaborolan-2-yl)phenyl) [¹⁴C]methanone

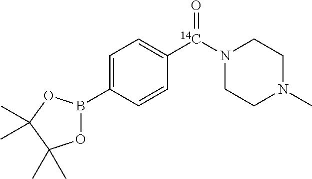

(4-Bromophenyl)(4-methylpiperazin-1-yl)[¹⁴C]methanone (269 mg, 0.94 mmol), bis(pinacolato)diboron (287 mg, 1.13 mmol), potassium acetate (370 mg, 3.77 mmol) and PdCl₂(dppf)-CH₂Cl₂ adduct (38.5 mg, 0.05 mmol) were mixed in dioxane (4 mL). Argon was bubbled through the solution for 1 min. The mixture was heated at 120° C. for 40 min in a microwave reactor. The mixture was diluted with dichloromethane and filtered. The solvents were evaporated and the mixture was purified by column chromatography on silica eluting with dichloromethane:methanol 10:1 to give the title compound (350 mg) that was used without further purification. MS (ESI⁺) m/z 333 [M+H]⁺.

### Example 17

### 4-(5-Amino-6-oxazolo[4,5-c]pyridin-2-yl-pyrazin-2-yl)[¹³C₆]phenyl]-(4-methylpiperazin-1-yl)methanone hydrochloride

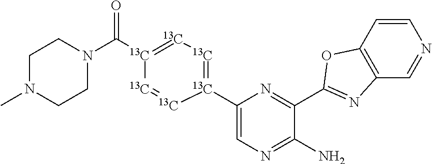

5-Bromo-3-(oxazolo[4,5-c]pyridin-2-yl)pyrazin-2-amine (0.190 g, 0.46 mmol), (4-methylpiperazin-1-yl)-[4-(4,4,5,5-tetramethyl-1,3,2-dioxaborolan-2-yl)[¹³C₆]phenyl]methanone (0.153 g, 0.46 mmol) and PdCl₂(dppf)-CH₂Cl₂ adduct (0.019 g, 0.02 mmol) were mixed in THF (3 mL). Sodium carbonate (2M, aq) (0.68 mL, 1.37 mmol) was added. Argon was bubbled through the solution for 1 min. The mixture was heated at 130° C. for 40 min in a microwave reactor. The mixture was diluted with dichloromethane. The mixture was filtered through a plug of diatomeous earth. The filter plug was washed with dihchloromethane and methanol. The solvents were evaporated and the mixture was purified by column chromatography on silica eluting with gradients of dichloromethane: methanol: ammonia in methanol and dichloromethane The fractions containing product were evaporated to give the title compound as the free base (98 mg, 51%). The residue was dissolved in methanol and dichloromethane. The mixture was filtered. HCl (1M in diethylether, 0.5 ml) was added. The mixture was left for 1 h. The solid was isolated by filtration and was dried under vacuum at 50° to give the title compound (50 mg, 0.109 mmol). More material could be recovered from the mother liquor.

¹H NMR (500 MHz, DMSO-d₆) δ ppm 2.80 (d, 3H) 3.11 (m, 2H) 7.46 (d, 1H) 7.78 (m, 1H) 8.05 (d, 1H) 8.14 (br. s., 1H) 8.21 (d, 1H) 8.37 (d, 1H) 8.78 (d, 1H) 9.07 (s, 1H) 9.37 (s, 1H) 10.73 (br. s., 1H)MS (APCI⁺) m/z 422 (M+H)⁺.

### i) (4-Bromo[¹³C₆]phenyl)(4-methylpiperazin-1-yl)methanone

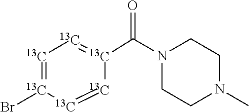

4-Bromo[¹³C₆]benzoic acid (0.100 g, 0.48 mmol) was dissolved in DMF (0.5 mL) and acetonitrile (2 mL). Hunig's Base (0.253 mL, 1.45 mmol), N-methylpiperazine (0.080 mL, 0.72 mmol) and O-benzotriazol-1-yl-tetramethyluronium hexafluorophosphate (0.220 g, 0.58 mmol) were added. The mixture was stirred at RT under argon atmosphere for 16 h. NaHCO₃ (aq) was added and the mixture was extracted with dichloromethane (×3). The organic phase was dried (Na₂SO₄) and concentrated. The residue was purified by column chromatography on silica eluting with gradients of dichloromethane/MeOH/7N ammonia 90/10/1 and dichloromethane. The fractions containing product were pooled and evaporated to give the title compound (152 mg, quant).

¹H NMR (500 MHz, CHLOROFORM-d) δ ppm 2.35-2.65 (m, 7H) 3.50 (br. s., 2H) 3.83 (br. s., 2H) 7.13 (m, 1H) 7.42 (m, 2H) 7.72 (m, 1H)

MS (ESI⁺) m/z 289 (M+H)⁺.

### ii) (4-Methylpiperazin-1-yl)[4-(4,4,5,5-tetramethyl-1,3,2-dioxaborolan-2-yl)[¹³C₆]phenyl]methanone

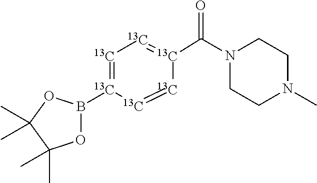

(4-Bromo[¹³C₆]phenyl)(4-methylpiperazin-1-yl)methanone (0.152 g, 0.53 mmol), bis(pinacolato)diboron (0.160 g, 0.63 mmol), potassium acetate (0.206 g, 2.10 mmol) and PdCl₂(dppf)-CH₂Cl₂ adduct (0.021 g, 0.03 mmol) were mixed in dioxane (3 mL) Argon was bubbled through the solution for 1 min. The mixture was heated 120° C. for 40 min in a microwave reactor. The mixture was diluted with dichloromethane. The mixture was filtered through a plug of diatomeous earth. The solvents were evaporated and the mixture was purified by column chromatography on silica eluting with gradients of dichloromethane: methanol: ammonia in methanol 90:10:1 0-100%. The fractions containing product were evaporated to give the title compound (0.153 g, 87%).

¹H NMR (500 MHz, CHLOROFORM-d) δ ppm 1.36 (s, 12H) 2.40 (br. s., 4H) 3.50 (s, 3H) 3.83 (br. s., 2H) 7.23 (m, 1H) 7.55 (m, 1H) 7.69 (m, 1H) 8.01 (m, 1H)

MS (ESI⁺) m/z 337 (M+H)⁺.

### General Methods

¹H NMR spectra were recorded in the indicated deuterated solvent at 400 MHz or 500 MHz. The 400 MHz spectra were obtained using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR or Bruker 500 MHz ultrashield spectrometer equipped with a 4-nucleus probehead with Z-gradients. Chemical shifts are given in ppm down- and upfield from TMS. Resonance multiplicities are denoted s, d, t, q, m and br for singlet, doublet, triplet, quartet, multiplet, and broad respectively.

LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 (5 μm, 50 mm×2 mm i.d) Flow: 1.0 mL/minGradient: 95% A to 100% B in 5 min. A=5% acetonitrile in water with 0.1% formic acid and B=acetonitrile with 0.1% formic acid. UV-DAD 210-400 nm Alternative, LC-MS analyses were recorded on a Waters 2790 LCMS equipped with a Phenomenex Luna C18 (5 μm, 50×4.6 mm i.d) The mobile phase system consisted of A: 10 mM ammonium formate(pH 4) in water and B: acetonitrile. A linear gradient was applied running from 95% to 5% B in 5 minutes with a flow rate of 2.0 mL/min. The mass spectrometer was equipped with an electrospray ion source (ESI) operated in a positive or negative ion mode. The capillary voltage was 3 kV and the mass spectrometer was typically scanned between m/z 100-700.

Mass spectra (MS) were run using an automated system with atmospheric pressure chemical (APCI or CI) or electrospray (+ESI) ionization. Generally, only spectra where parent masses are observed are reported. The lowest mass major ion is reported for molecules where isotope splitting results in multiple mass spectral peaks (for example when chlorine or bromine is present).

HPLC assays were performed using an Agilent HP1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min Mobile phase A: 10 mM ammonium acetate in water/acetonitrile (95:5), mobile phase B: acetonitrile. Alternatively the set up was Xbridge C8 30×50 mm 5 μm run at a flow rate of 2.0 ml/min. A linear gradient was applied, starting at 100% A (A: 10 mM NH₄OAc in 5% CH₃OH) and ending at 100% B (B: CH₃OH).

Preparative HPLC was performed on a Waters Auto purification HPLC-UV system with a diode array detector using a Waters XTerra® MS C₈ column (19×300 mm, 7 μm) with the gradient described.

The following general methods and instrumentation have been used for the compound in Example 1 (second method) and Method 4.

LC-MS analyses were recorded on a Waters Aquity UPLCMS equipped with a Acquity C18-column, (1.7 μm, 100 mm×2.1 mm i d) The mobile phase system consisted of A: 0.05% TFA in water and B: acetonitrile. A linear gradient was applied running from 10% to 90% B in 4-5 minutes with a flow rate of 0.25 mL/min. The mass spectrometer was equipped with an electrospray ion source (ESI) operated in a positive or negative ion mode. The capillary voltage was 3 kV and the mass spectrometer was typically scanned between m/z 100-700.

Alternative, LC-MS analyses were recorded on a Agilent 1200 LCMS equipped with a Zorbax SB C8 (3.5 μm, 150×4.6 mm i.d.) The mobile phase system consisted of A: 0.05% TFA in water and B: acetonitrile. A linear gradient was applied running from 10% to 90% B in 8 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700.

Mass spectra (MS) were run using an automated system with electrospray (+ESI) ionization. Generally, only spectra where parent masses are observed are reported. The lowest mass major ion is reported for molecules where isotope splitting results in multiple mass spectral peaks (for example when Bromine is present).

HPLC purities were performed using a Agilent UHPLC 1200 Series system equipped with a Aquity HSS T3, (100×2.1 mm, 1.8 μm) column. The column temperature was set to 40° C. and the flow rate to 0.6 mL/min. The Diode Array Detector was scanned from 200-300 nm. The mobile phase system comprise of A: 0.03% TFA in water and B: 0.03% TFA in acetonitrile. A gradient was applied according to the table 3 below:

TABLE 3

| Time (min) | % B |
|---|---|
| 0 | 5 |
| 4 | 15 |
| 8 | 90 |
| 9 | 90 |
| 9.1 | 5 |

¹⁴C-Compounds were identified by coelution with unlabelled reference material on analytical HPLC and TCL using UV and radiometric detection.

The compounds have been named using CambridgeSoft MedChem ELN v2.1, ACD/Name, version 8.08, software from Advanced Chemistry Development, Inc. (ACD/Labs), Toronto ON, Canada, www.acdlabs.com, 2004 or are according to IUPAC convention.

### Pharmacology

Determination of GSK3β activity in Scintillation Proximity Assay.

The inhibition experiments were carried out in duplicate with 10 concentrations of the inhibitor in 384 well clear-bottom microtitre plates. A biotinylated peptide substrate (Biotin-Ala-Ala-Glu-Glu-Leu-Asp-Ser-Arg-Ala-Gly-Ser(PO3H2)-Pro-Gln-Leu (AstraZeneca, Lund)), was added at a final concentration of 1 μmol/L in an assay buffer (pH 7.0) containing 0.6 mU recombinant human GSK3β (Dundee University, UK), (˜5 nmol/L active enzyme), 10 mmol/L morpholinepropanesulfonic acid (MOPS), 0.3 mmol/L EDTA, 0.01% (v/v) (3-mercaptoethanol, 0.003% (w/v) polyethylene 23 lauryl ether (Brij 35), 0.4% glycerol and 0.02 mg bovine serum albumine (BSA) and preincubated for 10 minutes. The reaction was initiated by the addition of 0.06 μCi [γ-33P]ATP (Amersham, UK) and unlabelled ATP in 30 mmol/L Mg(Ac)₂ to a final concentration of 1 μmol/L ATP. The final assay volume was 15 μL. Blank controls without peptide substrate were used. After incubation for 15 min at room temperature, the reaction was terminated by the to addition of stop solution containing 1.3 mmol/L EDTA, 13 μmol/L ATP, 0.02% Triton™X-100 and 0.15 mg streptavidin coated SPA beads. After a 5 minutes centrifugation at 2000 rpm, the radioactivity was measured in a liquid scintillation counter (1450 MicroBeta Trilux, Perkin Elmer, Finnland). The Km value of ATP for GSK3β, used to calculate the inhibition constants (Ki) of the various compounds, was 3 μM.

### Determination of the In Vitro Axl Kinase Assay.

The following assays can be used to measure the effects of the compounds of the present invention as inhibitors of Axl tyrosine kinase enzyme, as inhibitors in vitro of the phosphorylation of Axl expressed on NCI H1299 lung large cell carcinoma cells. The assay used AlphaScreen technology (Gray et al., Analytical Biochemistry, 2003, 313: 234-245) to determine the ability of test compounds to inhibit phosphorylation by recombinant Axl tyrosine kinase.

N-terminal GST-Axl kinase domain encompassing amino acids 473 to 894 of Axl (GenBank Accession No NM_{—}021913) was expressed in SF126 insect cells and purified using the GST epitope tag, using standard purification techniques.

Test compounds were prepared as 10 mM stock solutions in dimethylsulphoxide (DMSO) and diluted in DMSO as required. Aliquots (120 nl) of compound dilutions were filled into the wells of a Greiner 384-well low volume (LV) white polystyrene plate (Greiner Bio-one) using acoustic dispensing (Labcyte Echo 550). A 10 μl mixture of recombinant purified Axl enzyme, biotinylated peptide substrate (Biotin poly-GAT; CisBio, Catalogue No. 61GATBLB), 0.2 μM adenosine triphosphate (ATP) and a buffer solution [comprising 20 mM Tris-HCl pH 7.5 buffer, 0.01% v/v Tween, 5 mM dithiothreitol (DTT), 0.1 mM NaVO₃ and 10 mM manganese chloride] was incubated with the compounds at room temperature for 20 minutes.

Control wells that produced a maximum signal corresponding to maximum enzyme activity were created by using 100% DMSO instead of test compound. Control wells that produced a minimum signal corresponding to 100% inhibited enzyme were created by adding 10 μM of Staurosporine.

Each reaction was stopped by the addition of 5 μl of a mixture of 500 mM EDTA, 3 mg/ml bovine serum albumin (BSA) and 20 mM Tris-HCl pH 7.4 buffer containing 40 ng/μl AlphaScreen Streptavidin donor and anti-p-Tyr-100 acceptor beads (Perkin Elmer, Catalogue No. 6760620M). The resultant signals arising from laser light excitation at 680 nM were read using a Packard Envision instrument. The mean data values for each test compound concentration, 100% DMSO control wells and 100% inhibition control wells were used to generate a dose response curve from which the test compound's IC₅₀ value the concentration that inhibited 50% of kinase activity, was estimated. The Km value for ATP for AXL, used to calculate the inhibitory constant (Ki) from IC50 values was 0.2 μM.

### Results

For the compound of formula (I) wherein R¹ is methyl the mean GSKβ Kᵢ value is 2.3 nM and the mean AXL Kᵢ value is 113.

For the compound of formula (I) wherein R¹ is hydrogen the mean GSKβ Kᵢ value is 3.2 nM and the mean AXL Kᵢ value is 26.

These data show that the compound of the present invention possess selectivity for GSK3 kinase enzyme compared with AXL kinase enzyme.

The Following Abbreviations have been Used:

| | |
|---|---|
| APCI | Atmospheric Pressure Chemical Ionisation |
| ATP | Adenosine Triphosphate |
| AXL | Tyrosine protein kinase receptor UFO |
| BSA | Bovin Serum Albumin |
| BuLi | Buthyl Lithium |
| CDI | Carbonyldiimidazole |
| m-CPBA | m-Chloroperoxybenzoic acid |
| Cs₂CO₃ | Cesium carbonate |
| DCM | Dichloromethane |
| DIPEA | Diisopropylethylamine |
| DMAP | 4-(dimethylamino)pyridine |
| DMF | N,N-dimethylfomamide |
| DMSO | Dimethyl sulfoxide |
| Dppf | 1,1′-Bis(diphenylphosphino)ferrocene |
| DTT | Dithiothreitol |
| DVS | Dynamic Vapor Sorption |
| EDCI | 1-(3-Dimethylaminopropyl)-3-ethylcarbodiimide |
| EDTA | Ethylenediaminetetraacetic acid |
| ESI | Electrospray ionisation |
| GSK3 | Glycogen synthase kinase 3 |
| HBr | Hydrobromic acid |
| HBSS | Hank's Balanced Salt solution |
| HBTU | O-Benzotriazol-1-yl-N,N,N′,N′-tetra-methyluronium |
| | hexafluorophosphate |
| HCl | Hydrochloric acid |
| HEPES | 4-(2-Hydroxyethyl)-1-piperazineethanesulfonic acid |
| HOBt | Hydroxybenzotriazole |
| IPA | 2-propanol |
| MeOH | Methanol |
| MES | 2-(N-morpholino)ethanesulfonic acid |
| MOPS | Morpholinepropanesulfonic acid |
| MS | Mass spectroscopy |
| MW | Microwave |
| NaOH | Sodium hydroxide |
| NAHCO₃ | Sodium hydrocabonate |
| Na₂S₂O₃ | Sodium thiosulfate |
| NBS | N-Bromosuccinimide |
| NIS | N-Iodosuccinimide |
| PBS | Phosphate Buffered Saline |
| Pd₂dba₃ | Tris-(dibenzylideneacetone)dipalladium(0) |
| PMSF | Phenylmethylsulphonyl fluoride |
| RT | Room temperature |
| RH | Relative Humidity |
| SPA | Scintillation Proximity Assay |
| TBTU | O-Benzotriazol-1-yl-N,N,N′,N′-tetra-methyluronium |
| | tetrafluoroborate |
| THF | Tetrahydrofurane |
| TMEDA | N,N,N′,N′-Tetramethylethylendiamine |
| T₃P | 1-Propanephosphonic acid cyclic anhydride |
| TSTU | O-(N-succinimidyl)-N,N,N′,N′-tetra-methyluronium |
| | tetrafluoroborate |
| XRPD | X-Ray Powder Diffraction |
| h | hour |
| min | minute |

1. A compound of formula (I), or a pharmaceutically acceptable salt thereof 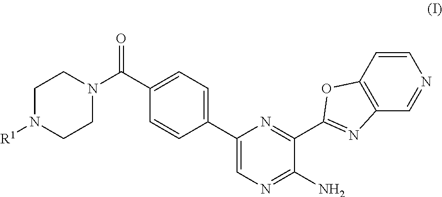
wherein R¹ is hydrogen or methyl.

2. A compound according to claim 1, or a pharmaceutically acceptable salt thereof, wherein R¹ is methyl.

3. A compound according to claim 1, which is (4-(5-amino-6-(oxazolo[4,5-c]pyridin-2-yl)pyrazin-2-yl)phenyl)(4-methylpiperazin-1-yl)methanone in the free base form.

4. A compound according to claim 1, which is (4-(5-amino-6-(oxazolo[4,5-c]pyridin-2-yl)pyrazin-2-yl)phenyl)(piperazin-1-yl)methanone in the free base form.

5. (canceled)

6. A pharmaceutical formulation comprising a compound of formula (I) or a pharmaceutically acceptable salt thereof according to claim 1 in association with a pharmaceutically acceptable adjuvant, diluent and/or carrier.

7-11. (canceled)

12. A method treating Type I diabetes, Type II diabetes, diabetic neuropathy; pain, neuropathic pain, nociceptive pain, chronic pain, pain associated with cancer, pain associated with rheumatic disease; alopecia; glaucoma; inflammatory diseases, inclusion body myositis; pemphigus vulgaris; benign or malignant tumours, non-solid tumours such as leukaemia including MLL leukemia; myeloma including multiple myeloma; or lymphoma; and solid tumours, for example bile duct, bone, bladder, brain/CNS, breast, colorectal, endometrial, gastric, head and neck, hepatic, lung, particularly non-small-cell lung, neuronal, oesophageal, ovarian, pancreatic, prostate, renal, skin, testicular, thyroid, uterine, vulval cancers; bone related effects of specific cancers for example breast, prostate, lung cancer, multiple myeloma, osteosarcoma, Ewing's sarcoma, chondrosarcoma, chordoma, malignant fibrous histiocytoma of bone, fibrosarcoma of bone, cancer induced bone disease, iatrogenic bone disease, benign bone disease, Padget's disease, osteoporosis, promoting bone formation; increasing cancellous bone formation and/or new bone formation, increasing bone mineral density, reducing the incidence of fracture and increasing the rate of fracture healing, comprising administering to a subject in need of such treatment, a therapeutically effective amount of a compound of formula (I) or a pharmaceutically acceptable salt thereof according to claim 1.

13. A compound of formula (VI) 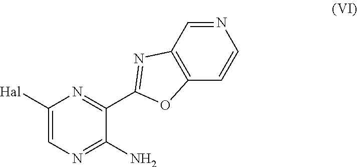
wherein Hal is chloro, bromo or iodo.

14. A compound according to claim 13, which is 5-bromo-3-(oxazolo[4,5-c]pyridin-2-yl)pyrazin-2-amine.

15. A pharmaceutical formulation comprising a compound of formula (I) or a pharmaceutically acceptable salt thereof according to claim 2 in association with a pharmaceutically acceptable adjuvant, diluent and/or carrier.

16. A pharmaceutical formulation comprising a compound of formula (I) or a pharmaceutically acceptable salt thereof according to claim 3 in association with a pharmaceutically acceptable adjuvant, diluent and/or carrier.

17. A pharmaceutical formulation comprising a compound of formula (I) or a pharmaceutically acceptable salt thereof according to claim 4 in association with a pharmaceutically acceptable adjuvant, diluent and/or carrier.

18. A method of treating early onset Alzheimer's Disease; presenile dementia of the Alzheimer's type; Alzheimer's Disease; Familial Alzheimer's disease; Early Alzheimer's disease; mild to moderate dementia of the Alzheimer's type or neurodegeneration associated with Alzheimer's disease, comprising administering to a subject in need of such treatment, a therapeutically effective amount of a compound according to claim 1.

19. A method of treating early onset Alzheimer's Disease; presenile dementia of the Alzheimer's type; Alzheimer's Disease; Familial Alzheimer's disease; Early Alzheimer's disease; mild to moderate dementia of the Alzheimer's type or neurodegeneration associated with Alzheimer's disease, comprising administering to a subject in need of such treatment, a therapeutically effective amount of a compound according to claim 2.

20. A method of treating early onset Alzheimer's Disease; presenile dementia of the Alzheimer's type; Alzheimer's Disease; Familial Alzheimer's disease; Early Alzheimer's disease; mild to moderate dementia of the Alzheimer's type or neurodegeneration associated with Alzheimer's disease, comprising administering to a subject in need of such treatment, a therapeutically effective amount of a compound according to claim 3.

21. A method of treating early onset Alzheimer's Disease; presenile dementia of the Alzheimer's type; Alzheimer's Disease; Familial Alzheimer's disease; Early Alzheimer's disease; mild to moderate dementia of the Alzheimer's type or neurodegeneration associated with Alzheimer's disease, comprising administering to a subject in need of such treatment, a therapeutically effective amount of a compound according to claim 4.

22. A method treating Type I diabetes, Type II diabetes, diabetic neuropathy; pain, neuropathic pain, nociceptive pain, chronic pain, pain associated with cancer, pain associated with rheumatic disease; alopecia; glaucoma; inflammatory diseases, inclusion body myositis; pemphigus vulgaris; benign or malignant tumours, non-solid tumours such as leukaemia including MLL leukemia; myeloma including multiple myeloma; or lymphoma; and solid tumours, for example bile duct, bone, bladder, brain/CNS, breast, colorectal, endometrial, gastric, head and neck, hepatic, lung, particularly non-small-cell lung, neuronal, oesophageal, ovarian, pancreatic, prostate, renal, skin, testicular, thyroid, uterine, vulval cancers; bone related effects of specific cancers for example breast, prostate, lung cancer, multiple myeloma, osteosarcoma, Ewing's sarcoma, chondrosarcoma, chordoma, malignant fibrous histiocytoma of bone, fibrosarcoma of bone, cancer induced bone disease, iatrogenic bone disease, benign bone disease, Padget's disease, osteoporosis, promoting bone formation; increasing cancellous bone formation and/or new bone formation, increasing bone mineral density, reducing the incidence of fracture and increasing the rate of fracture healing, comprising administering to a subject in need of such treatment, a therapeutically effective amount of a compound of formula (I) or a pharmaceutically acceptable salt thereof according to claim 2.

23. A method treating Type I diabetes, Type II diabetes, diabetic neuropathy; pain, neuropathic pain, nociceptive pain, chronic pain, pain associated with cancer, pain associated with rheumatic disease; alopecia; glaucoma; inflammatory diseases, inclusion body myositis; pemphigus vulgaris; benign or malignant tumours, non-solid tumours such as leukaemia including MLL leukemia; myeloma including multiple myeloma; or lymphoma; and solid tumours, for example bile duct, bone, bladder, brain/CNS, breast, colorectal, endometrial, gastric, head and neck, hepatic, lung, particularly non-small-cell lung, neuronal, oesophageal, ovarian, pancreatic, prostate, renal, skin, testicular, thyroid, uterine, vulval cancers; bone related effects of specific cancers for example breast, prostate, lung cancer, multiple myeloma, osteosarcoma, Ewing's sarcoma, chondrosarcoma, chordoma, malignant fibrous histiocytoma of bone, fibrosarcoma of bone, cancer induced bone disease, iatrogenic bone disease, benign bone disease, Padget's disease, osteoporosis, promoting bone formation; increasing cancellous bone formation and/or new bone formation, increasing bone mineral density, reducing the incidence of fracture and increasing the rate of fracture healing, comprising administering to a subject in need of such treatment, a therapeutically effective amount of a compound of formula (I) or a pharmaceutically acceptable salt thereof according to claim 3.

24. A method treating Type I diabetes, Type II diabetes, diabetic neuropathy; pain, neuropathic pain, nociceptive pain, chronic pain, pain associated with cancer, pain associated with rheumatic disease; alopecia; glaucoma; inflammatory diseases, inclusion body myositis; pemphigus vulgaris; benign or malignant tumours, non-solid tumours such as leukaemia including MLL leukemia; myeloma including multiple myeloma; or lymphoma; and solid tumours, for example bile duct, bone, bladder, brain/CNS, breast, colorectal, endometrial, gastric, head and neck, hepatic, lung, particularly non-small-cell lung, neuronal, oesophageal, ovarian, pancreatic, prostate, renal, skin, testicular, thyroid, uterine, vulval cancers; bone related effects of specific cancers for example breast, prostate, lung cancer, multiple myeloma, osteosarcoma, Ewing's sarcoma, chondrosarcoma, chordoma, malignant fibrous histiocytoma of bone, fibrosarcoma of bone, cancer induced bone disease, iatrogenic bone disease, benign bone disease, Padget's disease, osteoporosis, promoting bone formation; increasing cancellous bone formation and/or new bone formation, increasing bone mineral density, reducing the incidence of fracture and increasing the rate of fracture healing, comprising administering to a subject in need of such treatment, a therapeutically effective amount of a compound of formula (I) or a pharmaceutically acceptable salt thereof according to claim 4.
